(19) United States
(12) Patent Application Publication
(10) Pub. No.: US 2010/0286055 A1
(43) Pub. Date: Nov. 11, 2010

(54) USE OF WNT3A FOR ACCELERATION OF WOUND HEALING

(75) Inventors: Mark W. J. Ferguson, Manchester (GB); Hugh Laverty, Manchester (GB); Nicholas Occleston, Manchester (GB); Sharon O'Kane, Manchester (GB); Kerry Nield, Manchester (GB)

Correspondence Address: FOLEY AND LARDNER LLP;SUITE 500, 3000 K STREET NW, WASHINGTON, DC, 20007, US

(73) Assignee: RENOVO LIMITED

(21) Appl. No.: 12/527,185

(22) PCT Filed: Feb. 14, 2008
(86) PCT No.: PCT/GB2008/000495

§ 371 Date: Jun. 25, 2010

(30) Foreign Application Priority Data
Feb. 15, 2007 (GB) 0702929.1

(51) Int. Cl. A61K 38/17; A61P 17/02

(52) U.S. Cl. 514/18.6; 514/20.8; 514/21.2; 514/21.3

(57) ABSTRACT

Provided is WNT3A, or a therapeutically effective fragment or derivative thereof, for use as a medicament for accelerating wound healing. Also provided is a method of accelerating wound healing, the method comprising providing a therapeutically effective amount of WNT3 A, or a therapeutically effective fragment or derivative thereof, to a site where wound healing is to be accelerated. The medicaments and methods of the invention may be of particular use in accelerating the healing of skin wounds.

The present invention relates to medicaments for accelerating wound healing, and to methods of accelerating wound healing.

Wounds are a source of discomfort to those afflicted, and may be associated with, or give rise to, a number of clinical difficulties or complications.

The wound healing response proceeds through a number of overlapping processes, beginning with the inflammatory response, and proceeding via the production of granulation tissue, wound contraction, and reconstitution of a functional epithelial covering. Perturbation of any of these processes may retard or otherwise disrupt the normal healing response.

Wounds are painful, even aside from the events associated with their formation, and delays in the healing of wounds may be associated with extended incidences of pain to the sufferer. Wounds can also decrease the mechanical function of the injured area.

The continued presence of open wounds can also be associated with many clinical problems, including blood loss and possible incidence of infection.

In the light of the above, it will be seen that the acceleration of healing of wounds is advantageous for many different reasons. However, despite the fact that the advantages of accelerating wound healing are well recognised, there remains a paucity of medicaments and methods by which this aim may be achieved. Accordingly there is a requirement for new, alternative, and more effective, medicaments and methods by which acceleration of wound healing may be attained.

There are a number of adverse effects associated with current regimes used in the management of wounds. These include protracted healing times, which may ultimately lead to the development of chronic wounds. Other undesirable effects relate to the qualities of the replacement tissues or organs that are generated via the healing process.

The absence of a universally accepted method for accelerating the healing of wounds is indicative of the need for novel medicaments and methods by which such acceleration may be effected. It is well recognised that there are failings and disadvantages associated with many of the current therapies available. Even in the case of relatively successful therapies, there is scope for improvement in terms of increased efficacy, or other parameters.

The WNT family of genes (wingless-type MMTV integration site family) encode a number of proteins that function as pleiotropic cell signalling molecules. These proteins, designated WNTs, share a number of conserved residues, including a characteristic cysteine pattern. It is these structural features, rather than shared function, that define the WNT proteins, since the effects of various WNT family members may differ markedly depending on the responding cells.

It is generally believed that Frizzled (Fz) molecules constitute the primary group of receptors for WNT family members. Frizzled receptors comprise seven membrane-spanning portions as well as a long amino terminal region designated the cysteine-rich domain (CRD). The CRD appears to constitute the WNT-binding portion of Fz receptors. Effective WNT signalling requires not only the presence of WNT and a Fz receptor, but also the presence of a protein of the LRP (LDL receptor related protein) class.

WNT3A is a member of the WNT family of signalling molecules. Human WNT3A is a 352 amino acid polypeptide, the sequence of which is shown in Sequence ID No. 1. The human and murine forms of WNT3A share 96% amino acid identity. The sequence of DNA encoding human WNT3A (also designated WNT3A) is set out in Sequence ID No. 2. The amino acid sequence of the murine equivalent (designated Wnt3a) is set out in Sequence ID No. 3, and the sequence of DNA encoding murine Wnt3a is set out in Sequence ID No.4. The amino acid sequence of rat Wnt3a is set out as Sequence ID No. 5, and the sequence of DNA encoding rat Wnt3a is set out in Sequence ID No.6.

Previous reports indicate that WNT3A is able to signal through a number of receptors, or receptor complexes. WNT3A has been shown to interact with LRP5 and LRP6, as well as Frizzled 8 (FZD8). The nucleotide sequence of LRP5 is shown as Sequence ID No. 7, and the amino acid sequence of LRP5 shown as Sequence ID No. 8. The nucleotide sequence of LRP6 is shown as Sequence ID No. 9, and the amino acid sequence of LRP6 shown as Sequence ID No. 10. The nucleotide sequence of FZD8 is shown as Sequence ID No. 11, and the amino acid sequence of FZD8 shown as Sequence ID No. 12.

It is an object of certain aspects of the invention to provide alternative medicaments capable of accelerating wound healing. It is an object of certain aspects of the invention to provide improved medicaments capable of accelerating wound healing. It is an object of certain aspects of the invention to provide alternative methods of accelerating wound healing. It is an object of certain aspects of the invention to provide improved methods of accelerating wound healing.

In a first aspect, the present invention provides the use of WNT3A, or a therapeutically effective fragment or derivative thereof, in the manufacture of a medicament for accelerating wound healing. This aspect of the invention also provides WNT3A, or a therapeutically effective fragment or derivative thereof, for use as a medicament for accelerating wound healing.

In a second aspect, the invention provides a method of accelerating wound healing, the method comprising providing a therapeutically effective amount of WNT3A, or a therapeutically effective fragment or derivative thereof, to a site where wound healing is to be accelerated. The WNT3A, or therapeutically effective fragment or derivative thereof, may preferably be administered to the site where wound healing is to be accelerated. The site may preferably be a wound.

The present invention is based on the inventors' new and surprising finding that WNT3A, as well as suitable fragments or derivatives thereof, may be used to accelerate wound healing. Without wishing to be bound by any hypothesis, the inventors believe that the acceleration of wound healing observed occurs as a result of increased wound contraction, and the invention provides WNT3A, or a therapeutically effective fragment or derivative thereof, for use as a medicament for promoting wound contraction. There is nothing in the prior art that would previously have led the skilled person to believe that WNT3A, or its fragments or derivatives, may be used to accelerate wound healing.

The recognition that WNT3A, or therapeutically effective fragments or derivatives thereof, may be used to accelerate wound healing provides a new mode by which wound healing may be therapeutically accelerated. This finding also gives rise to the prospect of improved medicaments and methods by which wound healing may be accelerated.

It may be preferred that the medicaments or methods of the invention are used to accelerate healing of skin wounds.

It may be preferred that the medicaments or methods of the invention utilise WNT3A itself. The WNT3A to be used may preferably be human WNT3A, as set out in Sequence ID No. 1.

Various terms that are used in the present disclosure to describe the invention will now be explained further. The definitions provided below may be expanded on elsewhere in the specification as appropriate, and as the context requires.

“Therapeutically Effective Fragments or Derivatives of WNT3A”

For the purpose of the present disclosure, “therapeutically effective fragments or derivatives of WNT3A” should be taken (except for where the context requires otherwise) to encompass any fragment or derivative of WNT3A that is able to accelerate wound healing. Preferred means by which such acceleration may be assessed are considered elsewhere in the specification.

Except for where the context requires otherwise, it should be considered that therapeutically effective derivatives may be derived either from WNT3A itself, or from therapeutically effective fragments of WNT3A.

A therapeutically effective fragment or derivative of WNT3A may be a fragment or derivative that is effective to accelerate healing of a treated wound by at least 10% compared to the rate of healing of a comparable untreated or control wound. Preferably a therapeutically effective fragment or derivative of WNT3A may be capable of accelerating healing by at least a 20%, more preferably at least 50%, even more preferably at least 75% and yet more preferably of accelerating healing by at least 90% compared to the rate of healing of an untreated or control wound. A most preferred therapeutically effective fragment or derivative of WNT3A may be capable of accelerating the healing of a wound by 100% or more, compared to the rate of healing of an untreated or control wound.

In particular, therapeutically effective fragments or derivatives of WNT3A suitable for use in the medicaments or methods of the invention may be those able to decrease the area or width of a wound. Suitable therapeutically effective fragments or derivatives of WNT3A may be those capable of bringing about such a decrease in the width of a wound by increasing the rate of wound contraction, or by increasing the rate of granulation tissue formation.

Preferably a therapeutically effective fragment or derivative of WNT3A may be one that is capable of accelerating the healing of a wound to which the fragment or derivative of WNT3A is added. Suitable therapeutically effective amounts of WNT3A, as well as suitable therapeutically effective fragments or derivatives of WNT3A, are considered elsewhere in the specification.

WNT3A, or therapeutically effective fragments or derivatives thereof suitable for use in accordance with the present invention, should preferably be taken to exclude members of the WNT family other than WNT3A.

“Therapeutically Effective Fragments”

Therapeutically effective fragments of WNT3A suitable for use in accordance with the present invention may comprise 25 or more amino acid residues from Sequence ID No. 1, preferably up to 100 amino acid residues, more preferably up to 200 amino acid residues, and even more preferably up to 300 amino acid residues. Fragments suitable for use in the medicaments and methods of the present invention include those comprising up to 350 amino acids residues of Sequence ID No. 1. Preferred fragments will comprise at least 25 amino acid residues from Sequence ID No. 1.

Therapeutically effective fragments of WNT3A suitable for use in accordance with the present invention may comprise up to 10 contiguous amino acid residues from Sequence ID No. 1, preferably up to 100 contiguous amino acid residues, more preferably up to 200 contiguous amino acid residues, and even more preferably up to 300 contiguous amino acid residues. Fragments suitable for use in the medicaments and methods of the present invention include those comprising up to 350 amino acids residues of Sequence ID No. 1. Preferred fragments will comprise at least 10 contiguous amino acid residues from Sequence ID No. 1.

Therapeutically effective fragments of WNT3A suitable for use in accordance with the present invention may comprise at least 10 contiguous amino acid residues from Sequence ID No. 1, preferably at least 100 contiguous amino acid residues, more preferably at least 200 contiguous amino acid residues, and even more preferably at least 300 contiguous amino acid residues. Fragments suitable for use in the medicaments and methods of the present invention include those comprising at least 350 amino acids residues of Sequence ID No. 1.

As described elsewhere in the specification, preferred therapeutically effective fragments of WNT3A will be those that incorporate a receptor-binding region of WNT3A (either in whole or in part).

WNT proteins are generally palmitoylated on a cysteine residue. Studies in which palmitoylation of WNTs has been disrupted by acyl protein thioesterase indicate that the presence of palmitate is essential in order for WNTs to exert their biological activity.

The inventors believe that WNT3A is palmitoylated on the cysteine residue located at position 77 in the amino acid sequence shown in Sequence ID No. 1. Accordingly, it is preferred that fragments of WNT3A for use in accordance with the invention should be fragments that comprise the cysteine residue located at position 77 of Sequence ID No. 1 (the skilled person will readily appreciate that the numbered position of this cysteine residue, referred to as cysteine 77, may change within a particular fragment depending on the length of the fragment in question). Preferred fragments of WNT3A may be palmitoylated fragments, and particularly those palmitoylated at cysteine 77.

Preferred fragments may include amino acid residues involved in binding of WNT3A to its cellular receptors. Previous reports indicate that WNT3A is able to signal through a number of receptors, or receptor complexes. WNT3A has been shown to interact with both LRP5 and LRP6 as well as FZD8.

Preferred therapeutically effective fragments or derivatives of WNT3A will be those that incorporate a receptor-binding region of WNT3A (either in whole or in part). It will be appreciated that it is the three dimensional structure of WNT3A that is important in considering receptor binding, and that accordingly suitable fragments may be selected based upon their ability to assume the requisite three dimensional conformation necessary for receptor binding.

“Therapeutically Effective Derivatives”

Although peptides comprising all or part of WNT3A (as defined by Sequence ID No. 1) represent preferred agents for use in accordance with the present invention, it will be recognised that there are contexts in which the sensitivity of peptides to degradation may be disadvantageous. There are many known techniques by which peptide derivatives may be produced that have greater resistance to degradation than do the original peptides from which they are derived.

Peptoid derivatives may be expected to have greater resistance to degradation than do peptide agents of the invention, whilst retaining the same ability. Suitable peptoid derivatives may be readily designed from knowledge of WNT3A's sequence and structure. Commercially available software may be used to develop suitable peptoid derivatives according to well-established protocols. It will be appreciated that the therapeutic effectiveness of peptoid and other derivatives may be investigated using the same techniques that allow assessment of therapeutic effectiveness of peptide fragments.

Retropeptoids based on WNT3A or its therapeutically effective fragments (but in which all amino acids are replaced by peptoid residues in reversed order) are also able to accelerate wound healing. A retropeptoid may be expected to bind in the opposite direction in the ligand-binding groove, as compared to a peptide or peptoid-peptide hybrid containing one peptoid residue. As a result, the side chains of the peptoid residues are able to point in the same direction as the side chains in the original peptide.

D-amino acid forms of WNT3A or its therapeutically effective fragments also confer the requisite ability to accelerate wound healing. In the case of D-amino acid forms, the order of the amino acid residues comprising the derivative is reversed as compared to those in the original peptide. The preparation of derivatives using D-amino acids rather than L-amino acids greatly decreases any unwanted breakdown of such an agent by normal metabolic processes, decreasing the amounts of agent which need to be administered, along with the frequency of its administration.

It will be appreciated that derivatives suitable for use in the medicaments and methods of the invention clearly include both those derived from full length WNT3A and those derived from therapeutically effective fragments of WNT3A.

A therapeutically effective derivative of WNT3A suitable for use in accordance with the present invention may share at least 10% homology with Sequence ID No. 1, preferably at least 25% homology, more preferably at least 50% homology, and even more preferably at least 75% homology. Particularly preferred derivatives may share at least 80%, 85%, 90%, 95% or greater homology with Sequence ID No. 1.

Therapeutically effective derivatives of WNT3A suitable for use in accordance with the present invention may share at least 10% identity with Sequence ID No. 1, preferably at least 25% identity, more preferably at least 50% identity, and even more preferably at least 75% identity. Particularly preferred derivatives may share at least 80%, 85%, 90%, 95% or greater identity with Sequence ID No. 1.

Therapeutically Effective Amounts

A therapeutically effective amount of WNT3A, or a fragment or derivative thereof, is any amount of WNT3A, or a therapeutically effective fragment or derivative thereof, which is able to accelerate the healing of a wound. A therapeutically effective amount of WNT3A, or a fragment or derivative thereof, is preferably an amount of WNT3A, or a fragment or derivative thereof, which is able to accelerate the healing of a wound to which the WNT3A, or fragment or derivative, is administered.

A therapeutically effective amount of a medicament of the invention is any amount of a medicament of the invention which is able to accelerate the healing of a wound. This acceleration of healing may preferably be achieved in a wound to which the medicament of the invention is administered.

A therapeutically effective amount of WNT3A, or a fragment or derivative thereof, or a therapeutically effective amount of a medicament of the invention, may preferably be an amount that is effective to accelerate healing of a treated wound by at least 10% compared to the rate of healing of a comparable untreated or control wound. Preferably a therapeutically effective amount of WNT3A, or a fragment or derivative thereof, or of a medicament of the invention, may be an amount capable of accelerating healing by at least 20%, more preferably at least 50%, even more preferably at least 75% and yet more preferably of accelerating healing by at least 90% compared to the rate of healing of an untreated or control wound. A most preferred therapeutically effective amount of WNT3A, or a fragment or derivative thereof, or of a medicament of the invention, may be capable of accelerating the healing of a wound by 100% or more, compared to the rate of healing of an untreated or control wound.

The skilled person will appreciate that a fragment or derivative of WNT3A that has little inherent therapeutic activity will still be therapeutically effective if administered in a quantity that provides a therapeutically effective amount.

Guidance as to specific amounts of WNT3A, or its fragments or derivatives, that may represent therapeutically effective amounts suitable for use in the medicaments or methods of the invention, is provided elsewhere in the specification.

“Medicaments of the Invention”

For the purposes of the present disclosure, medicaments of the invention should be taken as encompassing any medicament manufactured in accordance with any aspect or embodiment of the invention. A medicament of the invention will generally constitute a preferred means for putting into practice any method of treatment in accordance with the present invention. Suitable compositions, formulations and routes of delivery that may be used for medicaments of the invention are considered elsewhere in the specification.

In particular, all references to “medicaments of the invention” should be taken to encompass medicaments manufactured in accordance with the first aspect of the invention, unless the context requires otherwise.

“Active Compound”

An “active compound”, for the purposes of the present disclosure, should be taken to be WNT3A, or any therapeutically effective fragment or derivative thereof. Active compounds should also be taken to encompass nucleic acids encoding WNT3A, or a therapeutically effective fragment or derivative thereof, and such nucleic acids represent active compounds that may be of particular use in gene therapy applications (as considered elsewhere in the specification).

“Topical Medicament”

A “topical medicament”, for the purposes of the present disclosure, is to be construed as a medicament that is applied at a site where it is intended to have its effect. This site may be a wound, or a site where a wound is to be formed. Topical medicaments suitable for use in accordance with the present invention include, but are not limited to, ointments; creams; lotions; gels; sprays; wound dressings capable of releasing active compounds to the body; and injectable solutions administered by local injections (e.g. intradermal injections).

“Wounds”

Except for where the context requires otherwise, references to “wounds” within the present disclosure should also be taken to encompass sites where wounds are to be formed, since the inventors have found that such sites may benefit from prophylactic treatment using the medicaments or methods of the invention.

For the purpose of the present disclosure wounds will primarily be described with reference to skin wounds, which comprise preferred wounds the healing of which may be accelerated in accordance with the present invention. However, the skilled person will appreciate that the acceleration of wound healing that may be achieved in accordance with the invention should not be limited to skin wounds. The inventors believe that wound healing may be accelerated, using the medicaments or methods of the invention, in wounds of all tissues.

Skin wounds, the healing of which may be accelerated using the medicaments and methods of the invention, include both chronic wounds and acute wounds. Examples of suitable chronic or acute wounds the healing of which may be accelerated in accordance with the invention are set out elsewhere in the specification.

Accordingly, it should be considered that the medicaments and methods of the invention may be used to accelerate healing of wound selected from the group consisting of abrasions; avulsions; crush wounds; incisional wounds; lacerations; punctures; ulcers, abscesses, and missile wounds, all of which may be suffered by the skin (among other tissues or organs). Further examples of suitable wounds, the healing of which may be accelerated using the medicaments or methods of the invention, include surgical wounds; pre-tibial lacerations; graft recipient sites; gastrointestinal ulcers, lung abscesses and wounds associated with myocardial infarction. Preferred chronic wounds that may be treated with the medicaments or methods of the invention in order to accelerate their healing include ulcers such as, diabetic ulcers, decubitus ulcers, and venous ulcers.

Examples of specific wounds, other than those of the skin, which may benefit from accelerated healing of wounds in accordance with the present invention include, but are not limited to, those selected from the group consisting of: wounds of the eye; wounds of blood vessels; wounds of the peripheral or central nervous system (where increasing the rate of healing of wounds may enhance the capability for neuronal reconnection); wounds of the oral cavity, including the lips and palate; wounds of the internal organs such as the liver, heart, brain and digestive tissues; and wounds in body cavities such as the abdominal cavity, pelvic cavity and thoracic cavity.

It is particularly preferred that the medicaments and methods of the invention be used to accelerate healing of skin wounds. This may be through treatment of such wounds themselves and/or the sites where such wounds are to be formed.

“Treated Wounds”, “Control-Treated Wounds” and “Untreated Wounds”

A “treated wound” in the context of the present disclosure is any wound that has been provided with a therapeutically effective amount of WNT3A (or a therapeutically effective fragment or derivative thereof) whether by a medicament of the invention, or in accordance with a method of treatment of the invention.

“Control-treated wounds” and “untreated wounds” in the present context are respectively wounds treated with a relevant control, and wounds that have not been treated before, or during, healing. Control wounds will not be treated with a medicament of the invention, and preferably will not be treated with a therapeutically effective amount of an active compound. That said, wounds treated with medicaments known from the prior art may constitute suitable control wounds for comparative purposes (for example to illustrate increased efficiency or effectiveness of medicaments of the invention as compared to those already known). A “diluent control-treated wound” will be an untreated wound to which a control diluent has been administered, and a “naïve control” will be an untreated wound made without administration of an active compound or a suitable control diluent, and left to heal without therapeutic intervention.

“Centimetre of Wound” and “Inch of Wound”

A “wound centimetre”, “centimetre of wound” or “centimetre of wounding” in the context of the present disclosure constitutes a unit by which the size of a wound to be treated may be measured.

A wound centimetre may be taken to comprise any square centimetre of a body surface that is wounded in whole or in part. For example, a wound of two centimetres length and one centimetre width (i.e. with a total surface area of two centimetres²) will also be considered to constitute “two wound centimetres”, while a wound having a length of two centimetres and a width of two centimetres (i.e. a total surface area of four centimetres²) will constitute four wound centimetres. By the same token, a linear wound of two centimetres length, but of negligible width (i.e. with negligible surface area), will, for the purposes of the present invention, be considered to constitute “two wound centimetres”, if it passes through two square centimetres of the body surface.

The size of a wound in wound centimetres should generally be assessed when the wound is in its relaxed state (i.e. when the body site bearing the wounded area is in the position adopted when the body is at rest). In the case of skin wounds, the size of the wound should be assessed when the skin is not subject to external tension.

An inch of wound may be similarly defined, save that the relevant units of length or area are measured in inches rather than centimetres.

A centimetre or inch of wounding may thus provide a unit by which the size of a wound to be treated may be measured, and the required amount of a medicament of the invention or of an active compound administered in accordance with a method of treatment of the invention) may be determined.

“Accelerating Wound Healing”

“Accelerating wound healing”, or “accelerating healing of wounds” in the context of the present disclosure should be taken to encompass any increase in the rate at which a wound heals.

Acceleration of wound healing achieved using the medicaments or methods of the invention may preferably lead to a treated wound healing at a rate at least 5% faster than an untreated or control wound, preferably at a rate at least 10% faster, more preferably at least 15%, 20% or 25% faster; yet more preferably at least 50% faster, still more preferably at least 75% faster, and most preferably 100% (or more) faster. The acceleration of healing may be brought about through promotion of wound contraction. Suitable methods by which promotion of contraction of wounds may be quantified to assess improvements in the rate of healing are described elsewhere in the specification.

Measurements that may be of use in assessing the rate of contraction of a wound include the rate at which the area or width of a wound decreases. Accelerated healing achieved using the medicaments or methods of the invention may preferably lead to a treated wound in which the wound's area or width decreases at a rate at least 5% faster than an untreated or control treated wound, preferably at a rate at least 10% faster, more preferably at least 15%, 20% or 25% faster; yet more preferably at least 50% faster, still more preferably at least 75% faster, and most preferably 100% (or more) faster. Such accelerated healing may be brought about by promotion of wound contraction. Suitable methods by which wound width may be measured in order to assess promotion of contraction of wounds are described elsewhere in the specification.

Acceleration of healing using the medicaments or methods of the invention may also give rise to a treated wound having an increased “healing age” when compared with an untreated or control treated wound. Such an increase in healing age may be assessed macroscopically, visually or clinically to determine maturity of the treated wound compared to a suitable untreated or control wound. A wound treated with the medicaments or methods of the invention may preferably have a healing age that is 1, 2, 3, 4, 5 or more days greater than that of an untreated, or control treated wound of the same chronological age.

Wounds to be Treated

As noted elsewhere, the skin suffers from more direct, frequent, and damaging encounters with the external environment than any other organ in the body. As a result the skin suffers from more wounds than other organs, and it is therefore highly desirable to be able to accelerate healing of skin wounds in order that the wounds may be closed and this organ returned as rapidly as possible to its normal functional effectiveness.

The medicaments or methods of the invention may be used to accelerate the healing of acute wounds or chronic wounds. Preferably, a wound the healing of which is to be accelerated using the medicaments of the invention may be selected from the group consisting of: superficial injuries, surgical wounds, abrasions, cuts, pressure ulcers stages i) to iv); venous stasis ulcers, ulcers caused by mixed etiologies; lower extremity ulcers; diabetic ulcers; radiation ulcers; arterial ulcers; partial thickness excisions; full thickness excisions; wounds in the immunocompromised, elderly or paraplegics; pre-tibial lacerations; wounds that have been debrided.

It will be appreciated that accelerated healing of wounds that may be achieved by the medicaments and methods of the invention may be of particular benefit in cases in which the wound healing response is impaired, inhibited, retarded or otherwise defective as compared to the normal rate of healing. The methods and medicaments of the invention may also be used to accelerate healing of wounds in patients that are not subject to an impaired healing response. Illustrative examples of both contexts are set out below.

There are many contexts in which the body's healing response is defective and may benefit from the acceleration of healing that may be achieved using the medicaments or methods of the invention. These include conditions such as pemphigus, Hailey-Hailey disease (familial benign pemphigus), toxic epidermal necrolysis (TEN)/Lyell's syndrome, epidermolysis bullosa, cutaneous leishmaniasis and actinic keratosis.

Healing of wounds may also be retarded as a result of the actions of pathogens (such as bacteria, fungi or viruses), chemical insults (such as chemical burns caused by caustic agents, or through the effect of cytotoxic drugs such as those employed in chemotherapy), or as a result of radiation damage (either through particulate radiation or electromagnetic radiation such as gamma radiation, ultraviolet radiation, or the like). Accordingly wounds subject to any of these influences may be particularly suitable subjects for accelerated healing using the medicaments or methods of the invention.

It is well known that dermal injuries in the aged heal more slowly than do those of younger individuals. The aged may therefore particularly benefit from accelerated healing that may be achieved using the medicaments and methods of the invention. There are also many other conditions or disorders that are associated with a delayed or otherwise impaired wound healing response. For example patients with diabetes, patients with polypharmacy (which may occur as a result of old age), post-menopausal women, patients susceptible to pressure injuries (for example paraplegics), patients with venous disease, clinically obese patients, patients receiving chemotherapy, patients receiving radiotherapy, patients receiving steroid treatment or immuno-compromised patients may all suffer from impaired healing. In some cases the slower healing response exhibited by such patients may contribute to the development of infections at the site of wounds. The slow wound healing response may also be associated with the formation of chronic wounds, as considered below. Accordingly, it will be appreciated that such patients represent a preferred group that may benefit from accelerated wound healing using the methods or medicaments of the invention.

Without detracting from the above, it may generally be preferred that the medicaments or methods of the invention may be used to accelerate healing of wounds of patients not subject to delayed wound healing. Accelerating healing in this way will give rise to a faster wound healing response than would normally be achieved by such patients (i.e. will give rise to faster healing than in control wounds). Accordingly the wounds of patients treated in this manner may be induced to heal more rapidly.

The skilled person will appreciate that there is a great benefit to be gained by society from the development of therapeutic agents and techniques that can hasten the healing of otherwise healthy patients. As well as the various benefits considered elsewhere in the specification, acceleration of wound healing in this manner can help reduce time spent in convalescence, and can thus benefit productivity. Accordingly, accelerating healing of wounds of healthy patients is a preferred embodiment of all aspects of the present invention.

The medicaments and methods of the invention may be used to accelerate the healing of both chronic wounds and acute wounds. For the purposes of the present invention, a chronic wound may be defined as any wound that does not show any healing tendency within eight weeks of formation when subject to appropriate (conventional) therapeutic treatment. Acute wounds may be any wound other than a chronic wound.

Accelerating the healing of chronic wounds is a preferred embodiment of the invention. Examples of chronic wounds that may benefit from accelerated healing provided by the medicaments or methods of the invention may be selected from the group consisting of: leg ulcers; venous ulcers; diabetic ulcers; bed sores; decubitus ulcers; foot ulcers; radiation ulcers; ulcers caused by mixed etiologies; and pressure ulcers. It will be appreciated that the long lasting nature of chronic wounds exacerbates many of the disadvantages associated with normal wound healing. For example, the duration of the period over which a patient suffering from a chronic wound will experience pain will generally be far longer than for a patient with an acute wound. Similarly the length of time over which desiccation as a result of liquid loss may occur will also be extended. Incidences of wound infection are also much increased in chronic, as opposed to acute, wounds. Accelerated healing using the medicaments or methods of the invention decreases the “open area” of treated wounds, and this may be of benefit in reducing the possible ingress of pathogens, and also reducing fluid loss from the damaged tissue.

Chronic wounds are also subject to many disadvantages that are not generally associated with acute wounds. For example, chronic wounds frequently expand beyond the limits of the original wounded area. This may arise as a result of infection (which may increase the damage around the margins of the wound, thereby leading to expansion) or through maceration of the tissue surrounding the wound (typically as a consequence of increased liquid loss through the chronic wound). The propensity for chronic wounds to expand beyond the boundary of the original injury means that such wounds are frequently of great surface area. The skilled person will appreciate that acceleration of wound healing using the medicaments or methods of the invention may be useful in reducing the area of chronic wounds, and may help to prevent the expansion of such wounds.

Pretibial lacerations are acute wounds of the leg that are very frequently slow to heal, and which frequently give rise to the development of leg ulcers. Existing treatments used for pretibial lacerations include the use of surgical procedures (such as the use of skin grafts and flaps) in an attempt to heal the wound before chronic wound development. Pretibial lacerations constitute acute wounds that may particularly benefit from treatment with the medicaments and methods of the invention, in order to accelerate their healing and reduce incidences of chronic wound formation.

It will be appreciated that tissues other than the skin may also be subject to wounds of the type described above and elsewhere in the specification. Such wounds may also benefit from the acceleration of healing that is provided by use of medicaments and methods of the invention.

Assessment of Acceleration of Wound Healing

A preferred measurement that may be used in assessing acceleration of wound healing is the rate at which the area of a wound decreases. Acceleration of wound healing using the medicaments or methods of the invention may preferably lead to a treated wound in which wound area decreases at a rate at least 5% faster than a control or untreated wound, preferably at a rate at least 10% faster, more preferably at least 15%, 20% or 25% faster; yet more preferably at least 50% faster, still more preferably at least 75% faster, and most preferably 100% (or more) faster.

An increase in the rate at which wound area decreases will indicate that healing of the wound in question has been accelerated. The rate at which the area of a treated wound decreases may be compared with control or untreated wounds, or with reference data regarding the rate at which areas of control or untreated wounds decreases in order to assess any differences in the rates observed.

A therapeutically effective amount of WNT3A, or a fragment or derivative thereof, in the context of the present invention may preferably be an amount of WNT3A, or a fragment or derivative thereof, that is able to give rise to a treated wound in which wound area decreases at a rate at least 5% faster than a control or untreated wound, preferably at a rate at least 10% faster, more preferably at least 15%, 20% or 25% faster; yet more preferably at least 50% faster, still more preferably at least 75% faster, and most preferably 100% (or more) faster.

The area of a wound may be assessed macroscopically or microscopically in order to determine the rate of wound healing. Suitable assessments of wound area may, for example, utilise photographs or tracings of the wound margins. These may be considered over time, or with reference to standard comparison data, to assess whether or not wound area is being therapeutically decreased.

The area of a wound assessed in this way should be distinguished from the degree of re-epithelialisation observed in the wound. Since the re-epithelialisation response will lead to overgrowth of the wound margins his may render assessment of wound area difficult. In general it may be preferred to assess wound area microscopically.

A preferred measurement that may be used in assessing acceleration of wound healing is the rate at which the width of a wound decreases. Acceleration of wound healing using the medicaments or methods of the invention may preferably lead to a treated wound in which wound width decreases at a rate at least 5% faster than a control or untreated wound, preferably at a rate at least 10% faster, more preferably at least 15%, 20% or 25% faster; yet more preferably at least 50% faster, still more preferably at least 75% faster, and most preferably 100% (or more) faster.

Suitable methods by which wound width may be measured in order to assess promotion of wound contraction are described elsewhere in the specification. It may generally be preferred that wound width be assessed microscopically, using histological slides. A preferred protocol for the assessment of wound width in full thickness wounds involves assessing the width of the wound at its mid point (i.e. a point mid way into the depth of the wound). The mid point is preferably in the dermis of the wound, well below the level at which re-epithelialisation occurs. Measurement of wound width at this point may avoid any inaccuracies that may otherwise be arise if wound width is not clearly distinguished from the portion of a wound that has not undergone re-epithelialisation.

The inventors believe that the increase in the rate at which width of treated wounds decreases is brought about primarily through an increase in wound contraction, and/or an increase in granulation tissue formation. Thus amounts of WNT3A (or a fragment or derivative thereof) capable of increasing wound contraction and/or granulation tissue formation represent preferred therapeutically effective amounts suitable for use in accordance with the present invention. It will also be appreciated that acceleration of wound healing may thus also be assessed with reference to the amount of wound contraction taking place, and alternatively or additionally, with reference to the amount of granulation tissue being formed.

Wound contraction is generally accepted to be dependent on the action of fibroblasts located both at the periphery of the wound and within the wound. Contraction is linked to fibroblast proliferation rate and connection of these cells to extracellular matrix components. The increase in the rate of wound contraction that may be achieved using the medicaments or methods of the invention should be distinguished from the rate at which the wound is covered with a new epithelial layer (in the skin a new epidermis), which is related to the rate of re-epithelialisation.

Granulation tissue formation arises primarily as a result of the influx of cells, such as fibroblasts, from the unwounded tissue surrounding the wound. The formation of granulation tissue plays an important role in filling the wound defect.

It may generally be preferred to use microscopic image analysis to measure the distance between the margins of the wound in the dermis, and thereby assess any wound contraction that may occur.

The inventors believe that treatment with the medicaments or methods of the invention gives rise to increased granulation tissue formation. Without being bound by any hypothesis, the inventors believe that this increased granulation tissue formation occurs as a result of increased cellular infiltration into the wound. This cellular infiltrate is resident in an immature ECM and is composed of many cell types including myofibroblasts. Fibroblasts and myofibroblasts among the cellular infiltrate are contractile and generate force across the wound that results in the contraction of the wound. Thus an increase in granulation tissue results in an increase in the number of contractile cells present in the wound resulting in a greater contractile force and thus promoting contraction of the wound and reducing its area (which appears as a narrowing of the wound width when assessed microscopically).

The skilled person will appreciate that increased wound contraction, and/or increased granulation tissue formation, are important in the acceleration of wound healing. These processes lead to the formation of narrower wounds, and ensure that wound size is rapidly decreased, as is the “open” area of the wound. The decrease in wound width may be particularly beneficial in the context of wounds that are to be healed by primary intention, since the margins of the relatively narrow wounds may be readily apposed (and then held in apposition by sutures, or the like). Accordingly it will be appreciated that the use of the medicaments or methods of the invention to promote the contraction of wounds that are to be healed by primary intention represents a preferred embodiment of the invention.

The width of a wound (and hence the rate at which the width of a wound decreases, and so the acceleration, or otherwise, of wound healing) may be assessed macroscopically, or microscopically.

In the case of microscopic assessment of wound width, this may be undertaken using suitable histological slides. Preferably wound width may be measured at a standardised “reference” point within the wound. The inventors have found that measurements taken midway through the depth of the wound allow for an accurate and reproducible assessment of wound width. Suitable image analysis software may aid the assessment of wound width in this manner.

Macroscopic assessment of wound width may either be performed directly (i.e. with measurements taken directly from a wound), or indirectly, in which case measurements may be taken using representations of the wound, such as photographs, traced outlines, mouldings, or the like. Image analysis software may be useful in the macroscopic assessment of wound width, particularly as assessed from photographs. In assessing wound width microscopically it is important that the width of a wound is differentiated from the degree of re-epithelialisation of the wound.

Given the potential for errors of measurement in macroscopic assessment of wound width it may generally be preferred to assess wound width microscopically rather than macroscopically.

Preferred Routes of Administration and Suitable Formulations

Preferred routes of administration, by which therapeutically effective amounts of WNT3A, or a fragment or derivative thereof, may be provided to a wound the healing of which it is desired to accelerate, are discussed more fully elsewhere in the specification. However, it may generally be preferred that WNT3A, or its fragments or derivatives, are provided by local administration to a wound, healing of which is to be accelerated. Suitable methods by which such local administration may be achieved will depend on the identity of the tissue in question, and may also be influenced by the size or location of the wound. Preferred routes of administration may include local injection (for example intradermal injection in the case where it is wished to accelerate healing of skin wounds). Other suitable means of administration include the use of topical medicaments such as sprays; powders; drops (e.g. for the ear or eye); ointments or creams; or release from local devices e.g. stents, implants, polymers, wound dressings, or the like.

Generally, medicaments of the invention may be formulated and manufactured in any form that allows for the medicament to be administered to a patient such that a therapeutically effective amount of the active compound is provided to a wound the healing of which is to be accelerated.

It will be appreciated that certain routes of administration normally associated with systemic administration may also be suitable for topical administration of active compounds to an otherwise “inaccessible” wound in which it is desired to accelerate healing (for example, inhalation or intranasal administration of active compounds may be of use in accelerating healing of wounds of the respiratory system).

Medicaments of the invention may preferably be provided in the form of one of more dosage units providing a therapeutically effective amount (or a known fraction or multiple of a therapeutically effective amount) of an active compound. Methods of preparing such dosage units will be well known to the skilled person; for example see Remington's Pharmaceutical Sciences 18^{th} Ed. (1990).

The medicaments of the invention should be taken to encompass any composition, material or device from which WNT3A, or a fragment or derivative thereof, may be provided to a wound in a therapeutically effective quantity (as defined elsewhere in the specification).

Examples of suitable compositions that may be utilised as medicaments of the invention include: creams, emulsions, ointments, irrigation solutions, sprays, foams, powders, gels, wound dressings, microneedles, liposomes, nanomicelles, thermosetting gels, microparticles, nanoparticles, and crystals.

In the case of suitable forms of medicaments of the invention that comprise a solid material, it may be preferred that the medicament be formulated such that a predetermined area of the medicament provides a therapeutically effective amount of an active compound to a wound to which the solid material is applied. This will be particularly beneficial in the case of solid medicaments, such as wound dressings, that are to be placed over a site where wound healing is to be accelerated.

Medicaments of the invention may be provided in the form of discrete dosage units capable of providing a therapeutically effective amount of an active compound. A suitable dosage unit in accordance with this embodiment of the invention may comprise a sufficient amount of a medicament of the invention to accelerate the healing of a given length or area of a wound.

A suitable dosage unit may comprise sufficient of a medicament of the invention to accelerate the healing of one centimetre of a wound. Alternatively a suitable dosage unit may comprise sufficient of a medicament of the invention to accelerate the healing of one inch of a wound. It will be appreciated that medicaments of the invention may be formulated to provide single dosage units or to provide multiple dosage units, as required. Thus a medicament of the invention may be packaged to provide one or more dosage units. Each dosage unit may provide a known fraction or multiple of a therapeutically effective amount of WNT3A, or a fragment or derivative thereof.

Suitable forms in which such discrete dosage units may be provided can be selected with reference to the nature of the medicament to be administered. Merely by way of example, medicaments of the invention comprising injectable solutions may be provided in the form of vials or pre-filled syringes comprising one or more dosage units. Other liquid medicaments in accordance with the invention, such as gels, creams, ointments, irrigation fluids or the like, may be provided in the form of tubes, sachets, cartons or blister packs comprising one or more dosage units.

Solid medicaments of the invention may readily be formulated such that a given area of the solid medicament is capable of providing sufficient WNT3A, or a fragment or derivative thereof, to accelerate the healing of a matching-sized area of wound. In such an embodiment a solid medicament of the invention may be cut to the required size and/or shape to cover a wound and the medicament will release a therapeutically effective amount of an active compound sufficient to accelerate the healing of the wound. Without wishing to be bound by any hypothesis, the inventors believe that an active compound released in this manner may be taken up via the wound, where the barrier function of the skin is impaired, but that the therapeutic effects of such a compound may actually occur as a result of its activity in the surrounding skin. Since the inventors believe that the therapeutic effects of WNT3A, or its therapeutically effective fragments or derivatives, may arise as a result of activity in the skin surrounding a wound to be treated, it will be appreciated that a solid medicament in accordance with the invention may alternatively be cut to a size that covers both a wound to be treated, and an area of normal skin surrounding the wound.

Solid formulations for use in medicaments of the invention may, or may not, be contained in a containment membrane or coating, microspheres, microgranules or microcapsules. The materials for such containment membranes or coatings may be selected from any of a variety of biodegradable natural or synthetic materials. Suitable materials may provide resistance to diffusion of the active compound.

It may be preferred that suitable materials for use in containment membranes or coatings be selected such that they allow sustained release of the active compound to the wound healing of which is to be accelerated. Examples of techniques by which this may be achieved will be well known to those skilled in the art, and will include the use of alternating layers of a suitable containment membrane or coating with layers of a carrier incorporating the active compound.

Suitable materials for use in containment membranes or coatings will generally degrade or be broken down over a period of time, thereby exposing the carrier, and allowing therapeutic release of the active compound from the carrier to the wound. The degradation or breakdown of suitable containment membranes or coatings may be caused by prolonged exposure to a wound. Factors that may mediate the degradation of such containment membranes or coatings will generally be the same as those that may cause the release of the active compound from the carrier. Such degradation or breakdown may typically be caused by moisture associated with the wound, or by the activity of enzymes active during wound healing.

Suitable solid formulations that may be used in medicaments of the invention may, for example, be selected from: powders; sprays, crystals; microneedles; solid compositions comprising microparticles, microspheres, microgranules, microcapsules, nanoparticles or liposomes; and wound dressings

Suitable liquid formulations that may be used in medicaments of the invention may, for example, be selected from: gels: thermosetting gels; creams; ointments; sprays; injectable solutions; irrigation solutions; other solutions of active compounds; and liquid compositions comprising microparticles, nanoparticles or liposomes.

In the case where the medicament of the invention is a wound dressing, suitable solid formulations may be applied throughout the dressing, and particularly to the surface of the dressing that is to be placed into contact with the wound. Solid formulations may be applied as a coating that may be applied to all the material of the dressing, or may be applied to discrete portions of the dressing (for instance the surface of the dressing that is to be placed in contact with the wound). Solid formulations may also be provided as granules, microgranules, microparticles, nanoparticles or liposomes adhered to the material of the dressing.

In the case of medicaments administered to sites of existing wounds, the wound will generally impair the skin's barrier function. As a result, this will normally facilitate the uptake of therapeutically effective amounts of the active compounds from medicaments of the invention without the need for permeation enhancers.

Preferably acceleration of wound healing using the medicaments or method of the invention may give rise to a healing time 1 day, 2 days, or 3 days faster than that occurring in a control-treated or untreated wound. Healing time may be calculated as the time elapsing between formation of a wound and complete closure of the wound (i.e. the point at which wound width becomes zero, or imperceptible). More preferably the acceleration of wound healing in accordance with the invention may give rise to a healing time that is at least 4 days, 5 days or 6 days faster than that occurring in a control-treated or untreated wound. It is even more preferred that acceleration of wound healing may give rise to a healing time that is at least 7 days, 8 days or 9 days faster than that occurring in a control-treated or untreated wound, and most preferably acceleration of wound healing may give rise to a time to wound closure that is at least 10 days or greater than that occurring in a control-treated or untreated wound.

The inventors have found that the medicaments and methods of the invention are able to promote accelerated wound healing when used either prior to the formation of a wound, or when used after a wound has already been formed. The use of the medicaments and methods of the invention prior to formation of a wound (in which case it is believed that the action of the medicament or method is to “prime” the site where wounding will occur so that accelerated healing is promoted immediately upon formation of the wound) is referred to as “prophylactic use” for the purposes of the present disclosure.

The prophylactic use of agents in accordance with the invention to accelerate wound healing is a preferred mode of use in accordance with the invention. It will be appreciated that such use is most suitable in the case where the time and location of prospective wound formation is known, and may be particularly suitable for accelerating healing of wounds associated with surgical procedures. However, prophylactic use of the medicaments or methods of the invention may also be of use in situations where there is an increased likelihood of wounding occurring. The inventors have found that administration of agents in accordance with the invention immediately prior to formation of a wound (e.g. in the hour preceding wounding, or preferably in the forty minutes or thirty minutes preceding wounding, and more preferably in the ten minutes preceding wounding) is highly effective, though administration at earlier times (e.g. up to 24 or 48 hours before wounding) may also beneficially accelerate wound healing. The prophylactic use of methods and medicaments of the invention is a preferred embodiment of the invention, and is particularly preferred in the event that it is wished to accelerate healing of surgical wounds.

Injection, and particularly intradermal injection, constitutes a preferred manner in which the medicaments of the invention may be administered (or the methods of the invention effected), as considered elsewhere in the specification. In the case of prophylactic use, it may be particularly preferred that a medicament of the invention be administered by intradermal injection to a site where wounding will take place. If the medicament is administered only a short time prior to wound, then intradermal injection of this type will typically lead to the formation of a raised bleb which will remain at the time of wounding. A wound may then be formed through the bleb. Wounds formed in this way will benefit from accelerated healing in accordance with the present invention. Alternatively, blebs formed by intradermal injection of medicaments of the invention may be allowed to resolve before a wound is formed.

The medicaments and methods of the invention may also be used to accelerate wound healing after a wound has been formed. This use will be the use generally adopted in respect of accidental wounds (and indeed most wounds formed other than in association with a surgical procedure).

When used to treat existing wounds medicaments in accordance with the invention may be applied along the margins of a wound to be treated. Preferably, medicaments of the invention may be injected along the margins of wounds to be treated. Injection in this manner also constitutes a preferred route of administration in accordance with the methods of treatment of the invention. In the case of skin wounds it is preferred that the route of injection selected is intradermal injection.

It will be appreciated that topical medicaments (other than topical injections) may also be applied to the margins of wounds the healing of which it is wished to accelerate. Alternatively, suitable medicaments may be administered to the wound defect itself, from where they may permeate the surrounding tissue, and thereby achieve their effect.

In the event that the medicaments or methods of the invention are to be used to accelerate healing of an existing wound, it is preferred that such use should occur as early as possible after formation of the wound. That said, the medicaments or methods of the invention may help to accelerate healing of a wound if used at any time up until the healing process is complete (for example even if administered to a partially healed wound the medicaments of the invention may usefully accelerate healing of the un-healed portion of the wound in a manner that will therapeutically decrease the time until the whole wound is fully healed).

A number of factors may be considered in determining the “window” in which medicaments or methods of the invention may be beneficially employed to accelerate healing of wounds. These may include:
i) the nature of the wound in question (for example: is the wound at a site that is generally subject to “fast” or “slow” healing?);
ii) the severity of the wound (what is the extent of the damage that has occurred?); and
iii) the size of the damaged area.

Thus, in the case of a wound of large area, or in a site that is naturally associated with slower than average healing, the methods or medicaments of the invention may still be effective to accelerate healing of the wound even if administered relatively late in the healing response. Thus, although the medicaments or methods of the invention may preferably be administered within the first one to 24 hours after formation of an acute wound, beneficial acceleration of healing may also be achieved if administered up to ten, or more, days after the wound is formed.

It will be appreciated that in the case of chronic wounds, the period in which the medicaments or methods of the invention may be beneficially employed will be considerably longer. Chronic wounds may persist for many years, and the healing of wounds that may be many years old may be beneficially accelerated using the medicaments or methods of the invention.

Therapeutic acceleration of wound healing may be achieved using only a single administration of the medicaments or methods of the invention. Due to the simplicity of this therapeutic regime it may constitute a preferred use of the medicaments and methods of the invention.

However, there may be cases in which it is preferred that the medicaments or methods of the invention be used in repeated incidences of therapy. Thus treatment to accelerate wound healing may involve administration of medicaments of the invention on more than once occasion. Use in this manner may be preferred in the case of large wounds, or of wounds that are resistant to treatment, or subject to retarded healing (such as chronic wounds). Generally medicaments of the invention may be administered to a wound as required until therapeutically effective acceleration of healing has been achieved (for example, until the wound has closed, or no further acceleration of healing may be achieved). By way of example, medicaments of the invention may be administered daily (or on multiple occasions within a given day), or may be administered after a delay of multiple days.

In a particularly preferred embodiment of the invention WNT3A, or a therapeutically effective fragment or derivative thereof, may be administered prior to formation of a wound, and then again after a wound has been formed. The inventors have found that this regime of treatment is particularly effective for the acceleration of wound healing, as demonstrated in the Experimental Results section. Both the first and second administrations of WNT3A, or a therapeutically effective fragment or derivative thereof, may be achieved utilising the medicaments of the invention. It may be preferred that the second administration of WNT3A takes place a day after formation of a wound the healing of which is to be accelerated.

Generally when the medicaments or methods of the invention are to be used in multiple therapeutic incidences, administration should be repeated until wound healing has been accelerated to a clinician's satisfaction.

It may be preferred that the medicaments or methods of the invention are utilised both before and after wounding.

It may be preferred that the medicaments of the invention are administered to a site where they are to have their effect around the time of wounding, or immediately prior to the forming of a wound (for example in the period up to six hours before wounding, and particularly in the period of 10, 20, 40 or 60 minutes prior to wound formation) or the medicaments may be administered at an earlier time before wounding (for example up to 48 hours before a wound is formed). The skilled person will appreciate that the most preferred times of administration prior to formation of a wound will be determined with reference to a number of factors, including the formulation and route of administration of the selected medicament, the dosage of the medicament to be administered, the size and nature of the wound to be formed, and the biological status of the patient (which may be determined with reference to factors such as the patient's age, health, and predisposition to healing complications) and the half-life of the active compound in the body, (where non-peptide derivatives of WNT3A may generally be expected to have longer half-lives than WNT3A or its fragments).

It may be particularly preferred that the methods or medicaments of the invention may be administered both before and after formation of a wound. The inventors believe that administration of an active compound prior to wound formation (and preferably immediately prior to the formation of a wound) followed by administration of an active compound on one or more days following wounding, is particularly effective in accelerating wound healing, through promotion of wound contraction

For the purposes of the present specification, an “agent” or “agent of the invention” will be WNT3A, or a therapeutically effective fragment or derivative thereof, or a compound able to promote expression of such an active compound. It will be appreciated that all such suitable agents may be incorporated in medicaments in accordance with the invention, and all may be used in the methods or uses of the invention. The medicaments of the invention represent preferred compositions by which a therapeutically effective amount of an active compound may be administered in order to put the methods of the invention into practice.

WNT3A (as shown in Sequence ID No 1) is a preferred example of an agent, or agent of the invention, as considered elsewhere in the specification.

It will be appreciated that the amount of a medicament of the invention (and hence an agent of the invention) that should be provided to a wound in order that a therapeutically effective amount of an active compound may be administered, depends on a number of factors. A number of these are discussed elsewhere in the specification, and include the biological activity and bioavailability of the agent present in the medicament, which in turn depends, among other factors, on the nature of the agent and the mode of administration of the medicament. Other factors in determining a suitable therapeutic amount of a medicament may include:
A) The half-life of the agent in the subject being treated.
B) The specific wound to be treated (e.g. accelerating healing of an acute wound or a chronic wound).
C) The age of the subject.
D) The size of the wound to be treated.

The frequency of administration will also be influenced by the above-mentioned factors and particularly the half-life of the chosen agent within the subject being treated.

Frequency of administration will depend upon the biological half-life of the agent used. Typically a cream or ointment containing an agent of the invention should be administered to a target tissue such that the concentration of the agent at a wound is maintained at a level suitable to accelerate wound healing. This may be achieved by a single administration of a composition incorporating an agent of the invention or may require administration of such a composition daily or even several times daily.

Medicaments of the invention, may be administered by any suitable route capable of achieving the desired effect of accelerating wound healing, but it is preferred that the medicaments be administered such that an agent of the invention is provided locally at a wound site or site where a wound is to be formed.

Administration (and particularly topical administration) of the medicaments of the invention may be effected as part of the initial and/or follow up care for the wounded area.

As suggested elsewhere in the specification, the agents of the invention may be provided on a dressing or patch, which may be used to cover a wound, the healing of which is to be accelerated. It will be appreciated that such a dressing or patch used to administer an agent of the invention may preferably be provided in a sterile form.

The agents of the invention may be released from a device or implant, or may be used to coat such a device e.g. a stent or controlled release device.

It will be appreciated that the vehicle of a composition comprising agents of the invention should be one that is well tolerated by the patient and allows release of the agent to a wound to which the composition is provided. Such a vehicle is preferably biodegradable, biocompatible, bioresolveable, bioresorbable and/or non-inflammatory. If the composition is to be applied to an existing wound then the pharmaceutically acceptable vehicle will be one that is relatively “mild”.

An agent of the invention may be incorporated within a slow or delayed release device. Such devices may, for example, be placed on or inserted under the skin and the agent may be released over days, weeks or even months. Such use may be of particular benefit when the selected agent is a nucleic acid encoding an active compound, or in the case of active compounds prone to degradation.

Delayed release devices may be particularly useful for patients requiring protracted treatment with the medicaments or methods of the invention, such as those requiring accelerated healing of chronic wounds. Delayed release devices may be particularly advantageous when used for the administration of an agent or nucleic acid that would otherwise normally require frequent administration (e.g. at least daily administration by other routes).

Daily doses of an agent of the invention may be given as a single administration (e.g. a daily application of a topical formulation or a daily injection). Alternatively, the agent of the invention may require administration twice or more times during a day. Each such administration may provide a therapeutically effective amount of the agent, or a known fraction of such a therapeutically effective amount. In a further alternative, a slow release device may be used to provide optimal doses of an agent of the invention to a patient without the need to administer repeated doses.

A dose of a composition comprising agents of the invention may preferably be sufficient to provide a therapeutically effective amount of an active compound in a single administration. However, it will be appreciated that each dose need not in itself provide a therapeutically effective amount of an agent or an active compound, but that a therapeutically effective amount of an agent or active compound may instead be built up through repeated administration of suitable doses.

Various suitable forms are known for compositions comprising agents of the invention. In one embodiment a pharmaceutical vehicle for administration of an agent of the invention may be a liquid and a suitable pharmaceutical composition would be in the form of a solution. In another embodiment, the pharmaceutically acceptable vehicle is a solid and a suitable composition is in the form of a powder or tablet. In a further embodiment the agent of the invention may be formulated as a part of a pharmaceutically acceptable patch or other transdermal delivery device capable of providing an agent of the invention to a wound or a site where a wound is to be formed.

A solid vehicle can include one or more substances that may also act as flavouring agents, lubricants, solubilizers, suspending agents, fillers, glidants, compression aids, binders or tablet-disintegrating agents; it can also comprise an encapsulating material. In powders, the vehicle is a finely divided solid that is in admixture with the finely divided agent of the invention. In tablets, the agent of the invention is mixed with a vehicle having the necessary compression properties in suitable proportions and compacted in the shape and size desired. The powders and tablets preferably contain up to 99% of the agent of the invention. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles may be used in preparing solutions, suspensions, emulsions, syrups, elixirs and pressurized compositions. The agent of the invention can be dissolved or suspended in a pharmaceutically acceptable liquid vehicle such as water, an organic solvent, a mixture of both, or pharmaceutically acceptable oils or fats. The liquid vehicle can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). Phosphate buffered saline (PBS) is a particularly preferred vehicle for use in compositions comprising WNT3A as the agent of the invention. For parenteral administration, the vehicle can be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by, for example, intramuscular, intrathecal, epidural, intraperitoneal, intradermal, intraadventitial (blood vessels) or subcutaneous injection. Sterile solutions can also be administered intravenously. The agent of the invention may be prepared as a sterile solid composition that may be dissolved or suspended at the time of administration using sterile water, saline, or other appropriate sterile injectable medium, such as PBS. Vehicles are intended to include necessary and inert binders, suspending agents, lubricants and preservatives.

Agents of the invention may be used to accelerate healing of “internal” wounds (i.e. wounds occurring within the body, rather than on an external surface of the body). Examples of internal wounds include penetrative wounds that pass through the skin into underlying tissues, and wounds associated with surgical procedures conducted within the body.

It will be appreciated that the use of medicaments or methods of the invention to accelerate healing of internal wounds will necessitate the use of suitable routes of administration, thereby requiring the formulation of the agents of the invention in a manner that allows their delivery to the wound in question. For example, medicaments in accordance with the invention for accelerating healing of wounds in the lungs or other respiratory tissues may be formulated for inhalation. In another preferred embodiment, medicaments in accordance with the invention for accelerating healing of wounds in the body cavities (such as the abdomen or pelvis) may be formulated as a lavage, gel or instillate.

Known procedures, such as those conventionally employed by the pharmaceutical industry (e.g. in vivo experimentation, clinical trials etc), may be used to establish specific formulations of compositions comprising agents of the invention and precise therapeutic regimes for administration of such compositions (such as daily doses of the active compound and the frequency of administration).

The inventors have observed that the acceleration of wound healing brought about by WNT3A (or its therapeutically effective fragments or derivatives) increases in relation to the increasing amount of the active compound provided to a wound. Based on this observation, the inventors believe that the amount of WNT3A that may be administered to a wound in order to therapeutically accelerate its healing may comprise up to approximately 750 ng per wound centimetre.

The inventors believe that a therapeutically effective amount of WNT3A may preferably be between 0.1 ng and 750 ng per wound centimetre, more preferably between 0.5 ng and 500 ng per wound centimetre, even more preferably between 1 ng and 250 ng per wound centimetre, and still more preferably between 1 ng and 100 ng per wound centimetre.

A most preferred therapeutically effective amount of WNT3A that may be used to accelerate healing in accordance with the present invention may be approximately 100 ng of WNT3A per wound centimetre.

It will be appreciated that therapeutically effective fragments or derivatives of WNT3A may have weights that differ significantly from that of WNT3A. In these cases, guidance as to suitable therapeutically effective amounts of the fragments or variants to be employed in the medicaments or methods of the invention may be derived from the numbers of moles of WNT3A provided by the therapeutically effective amounts of WNT3A considered above.

Thus, a therapeutically effective amount of WNT3A, or a fragment or derivative thereof, may comprise up to 18 pmoles of the active compound per wound centimetre. A therapeutically effective amount of WNT3A, or a fragment or derivative, may preferably comprise between approximately 2 fmoles and 18 pmoles per wound centimetre, more preferably between approximately 12 fmoles and 12 pmoles per wound centimetre, even more preferably between approximately 24 fmoles and 6 pmoles per wound centimetre, and still more preferably between approximately 24 fmoles and 2.4 pmoles per wound centimetre. Most preferably, a therapeutically effective amount of WNT3A, or a fragment or derivative thereof, may comprise approximately 2.4 pmoles per centimetre of wound.

A therapeutically effective amount of WNT3A, or a fragment or derivative thereof, may be provided in a solution at a concentration of up to 183 nM. A therapeutically effective amount of WNT3A, or a fragment or derivative, may preferably be provided in a solution at a concentration of between approximately 0.024 nM and 183 nM, more preferably in a solution at a concentration of between approximately 0.12 nM and 122 nM, even more preferably in a solution at a concentration of between approximately 0.24 nM and 61 nM, and still more in a solution at a concentration of between approximately 0.24 nM and 24 nM. Most preferably, a therapeutically effective amount of WNT3A, or a fragment or derivative thereof, may be provided in the form of solution at a concentration of approximately 24 nM.

In the event that a fragment or derivative of WNT3A comprises a different numbers of receptor binding sites to the number of receptor binding sites found in native WNT3A, this may alter the number of moles of such a fragment or derivative required in order to provide a therapeutically effective amount. For example, in the event that a derivative of WNT3A comprises twice the number of binding sites present in native WNT3A, the amount of the derivative that will be needed to provide a therapeutically effective amount will generally be half of the amount(s) suggested above. Other such variations will be readily apparent to the skilled person.

It will be recognised that the values suggested in the preceding paragraphs as suitable for application to a centimetre of wound will also generally be suitable for application to a centimetre of a site where a wound is to be formed in accordance with the prophylactic use of the medicaments or methods of the invention.

The skilled person will appreciate that the suggestions above are provided for guidance. In particular it will be appreciated that the amount of an active compound or agent of the invention to be provided via topical administration may be altered depending on permeability of the tissue or organ to which the topical composition is administered. Thus in the case of relatively impermeable tissues or organs it may be preferred to increase the amount of the agent administered. Such an increased amount of WNT3A, or a fragment or derivative thereof, may still represent a therapeutically effective amount, if the amount of the agent taken up into the tissue or organ where wound healing is to be accelerated is therapeutically effective (i.e. if a therapeutically effective amount permeates the tissue or organ where wound healing is to be accelerated, irrespective of the fact that a larger amount of the agent may remain on the surface of, and unable to penetrate, the tissue or organ being treated).

In a particularly preferred embodiment, WNT3A may be administered as a 24.4 nM solution. 100 μL of such a solution administered per centimetre of wound over a 24 hour period. WNT3A may be administered by way of an injectable solution containing approximately 100 ng/100 μL in order to accelerate wound healing. In particular such a solution may be administered as an intradermal injection providing 100 μL of solution per cm of wound.

It will be appreciated that the guidance as to doses and amounts of agents of the invention provided above is applicable both to medicaments of the invention, and also to the methods of the invention.

In the case where the paragraphs above consider the administration of a specified amount of a medicament per centimetre of a wound it will be appreciated that this volume may be administered to either one or both of the margins of a wound to be treated (i.e. in the case of a reference to 100 μl of a medicament, this may be administered as 100 μl along one of two wound margins to be joined together, or as 50 μl to each of the wound margins to be joined together).

Medicaments or methods of the invention may be used to accelerate wound healing as a monotherapy (e.g. through use of medicaments or methods of the invention alone). Alternatively the methods or medicaments of the invention may be used in combination with other compounds or treatments for the acceleration of wound healing. Suitable compounds that may be used as parts of such combination therapies will be well known to those skilled in the art.

The skilled person will appreciate that therapeutically effective amounts of WNT3A, or its fragments or derivatives, may be administered at the sites of wounds (or sites where wounds are to be formed) where it is wished to accelerate healing by virtue of cellular expression (commonly referred to as gene therapy). Accordingly, the invention provides a method of accelerating wound healing, the method comprising inducing cellular expression of a therapeutically effective amount of WNT3A, or a fragment or derivative thereof, at a site where healing is to be accelerated. Such a site may be a wound, or a site where a wound is to be formed, or where there is a heightened likelihood of wound formation occurring.

Based on the teaching contained in the present specification, it will be a matter of routine experimentation for one skilled in the art to devise protocols by which cells may be induced to express therapeutically effective amounts of WNT3A (or its fragments or derivatives).

For example, the skilled person will appreciate that such cellular expression of therapeutically effective amounts of WNT3A may be achieved by manipulating naturally occurring expression of this molecule by cells in the region of the wound to be treated.

Alternatively, and preferably, cells in the region of wound (or site where a wound is to be formed) that is to be treated may be induced to express WNT3A, or therapeutically effective fragments or derivatives thereof, by means of the introduction of materials encoding such agents. Suitable materials may typically comprise nucleic acids such as DNA or RNA, and these may be devised based upon the sequences referred to in this specification.

Nucleic acids for use in this embodiment of the invention may be administered “as is”, for example by means of ballistic transfection, or as parts of a larger construct, which may be able to incorporate stably into cells so transfected. Suitable constructs may also contain regulatory elements, by which expression of a therapeutically effective amount of WNT3A, or a fragment or derivative thereof, may be achieved. Such constructs give rise to further aspects of the present invention.

Thus the invention also provides a construct encoding WNT3A, or a therapeutically effective fragment or derivative thereof, said construct being capable of expression at a site where wound healing is to be accelerated to give rise to a therapeutically effective amount of the WNT3A, or therapeutically effective fragment or derivative. The invention also provides a method of accelerating wound healing, the method comprising administering a construct (as described above) to a site where wound healing is to be accelerated such that a therapeutically effective amount of WNT3A, or a therapeutically effective fragment or derivative thereof, is expressed. The invention also provides the use of such a construct in the manufacture of a medicament for the acceleration of wound healing.

It will be appreciated that many of the advantages that may be gained as a result of accelerated healing of human wounds are also are also applicable to wounds in other animals, particularly veterinary or domestic animals (e.g. horses, cattle, dogs, cats etc). Accordingly it will be recognised that the medicaments and methods of the invention may also be used accelerate the healing of wounds of non-human animals. Generally the same active compounds that may be used to accelerate healing of human wound may also be used in such cases, however it may be preferred to use WNT3A (or a therapeutically effective fragment or derivative thereof) that is derived from the same type of animal as is being treated (e.g. in the case of treatment of horses, use of equine WNT3A).

The invention will now be further described with reference to the accompanying Experimental Results and Figures, in which:

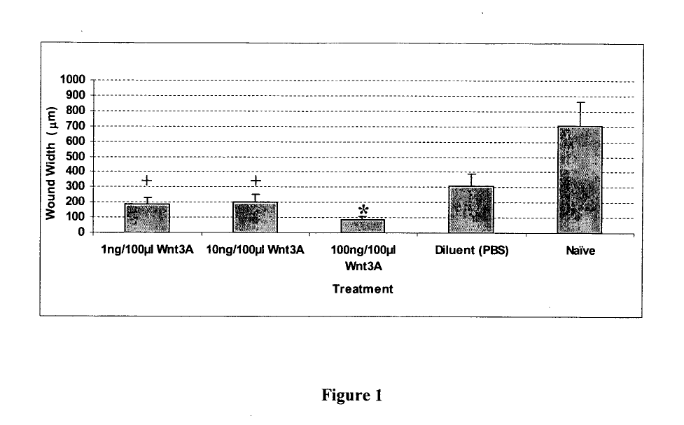

FIG. 1 is a bar chart comparing the wound widths of control (both diluent control and naïve control) and treated incisional wounds “+” indicates p<0.05 versus naïve controls only; “*” indicates p<0.05 versus both naïve control wounds and diluent control wounds. The amino acid sequence of human WNT3A (Sequence ID No. 1); the sequence of DNA encoding human WNT3A (Sequence ID No. 2); the amino acid sequence of murine Wnt3a (Sequence ID No. 3); the sequence of DNA encoding murine Wnt3a (Sequence ID No. 4); the amino acid sequence of rat Wnt3a (Sequence ID No. 5); the sequence of DNA encoding murine Wnt3a (Sequence ID No. 6) a comparison of the amino acid sequences of human WNT3A and murine WNT3A; a comparison of the amino acid sequences of human WNT3A and rat WNT3A; the nucleotide sequence of human LRP5 (Sequence ID No. 7); the amino acid sequence of human LRP5 (Sequence ID No. 8); the nucleotide sequence of human LRP6 (Sequence ID No. 9); the amino acid sequence of human LRP6 (Sequence ID No. 10); the nucleotide sequence of human Frizzled 8 (Sequence ID No. 11) and the amino acid sequence of human Frizzled 8 (Sequence ID No. 12) are all shown in the section entitled “Sequence Information”.

### EXPERIMENTAL RESULTS

The inventors investigated the ability of WNT3A to accelerate wound healing using in an in vivo model of healing.

Incisional Wound Healing Model and Treatment with WNT3A

Murine WNT3A (Catalogue number 1324-WN/CF, Lot HTR054051) was purchased from R&D Systems.

The WNT3A was diluted in phosphate buffered saline (PBS) to produce three solutions having concentrations as follows:
1. 1 ng/100 μl (a 0.24 nM solution);
2. 10 ng/100 μl (a 2.4 nM solution); and
3. 100 ng/100 μl (a 24.4 nM solution).

PBS alone was used as a diluent control.

Wounding Model, Dosing & Harvest Timepoint

At day 0, Male Sprague Dawley rats (200-250 g) were anaesthetised, shaved and wound sites were marked according to the following wounding template: 2×1 cm wounds incisional wounds formed 5 cm from the base of the skull and 1 cm from the midline of each rat. One hundred microlitres of WNT3A incorporated in the solutions described above (1 ng, 10 ng or 100 ng of WNT3A in 100 μl of PBS), were injected intradermally at the sites where wounds were to be formed. The intradermal injections caused the formation of a raised bleb, which was then immediately incised to form 1 cm long full thickness experimental wounds. A separate group of rats were wounded, without any injection, to act as the untreated naïve control group in addition to a group receiving diluent control injections (100 μl of PBS alone, without WNT3A).

Accordingly, each injection of the 1 ng/100 μl solution provided 24.4 fmoles of WNT3A, whilst each injection of the 10 ng/100 μl solution provided 244 fmoles of WNT3A, and each injection of the 100 ng/100 μl solution provided 2.4 pmoles of WNT3A.

All wounds receiving either treatment or diluent control injections were re-injected again 1 day post-wounding with the appropriate solution via injection of 50 μl to each of the two margins of the 1 cm wound. Wounds were then harvested at day 3 post-wounding.

The wounds were photographed after wounding, prior to re-injection on day 1 and on day of harvest. The wounds were analysed microscopically, by image analysis, to measure wound width in order assess the ability of the medicaments of the invention to accelerate wound healing.

Assessment of Wound Width

The widths of experimental wounds (treated wounds, diluent control wounds or naïve control wounds) were assessed in order to investigate the ability of the medicaments of the invention to promote wound contraction, and thereby accelerate wound healing.

Wounds were excised from the experimental animals (incorporating a small amount of surrounding normal tissue) and fixed in 10% (v/v) buffered formal saline. Both the anterior and posterior halves of the wound were processed to wax but only the anterior half was cut to produce tissue sections. Five slides were prepared from each wound, each slide comprising four, 5 μm thick, serial sections. One slide from each wound was stained with Haematoxylin and Eosin (H & E) and from this wound width measurements were taken. The width of each wound was measured at the mid-wound point (midway drought the depth of the wound, at the level of the dermis), the relevant measurements being made using image analysis equipment and software.

A comparison of the widths of WNT3A treated incisional wounds, incisional wounds injected with a diluent control, and naïve control incisional wounds, all measured at three days after wounding, is shown in FIG. 1.

Results

Acceleration of Wound Healing

Administration of medicaments of the invention (incorporating WNT3A) was able to accelerate the healing of wounds, as assessed by measurement of mid-wound width of incisional wounds three days after wound formation, at all concentrations investigated. The degree of acceleration of wound healing observed increased with the amount of WNT3A administered.

Administration of WNT3A at a concentration of either 1 ng/100 μl or 10 ng/100 μl (respectively equivalent to a total dose of 24.4 fmoles or 244 fmoles of WNT3A per cm of wound) significantly decreased wound width, as compared to naïve controls (p<0.05), when incisional wounds were assessed at three days after wounding. Administration of WNT3A at a concentration of 100 ng/100 μl (equivalent to a total dose of 2.4 pmoles of WNT3A per cm of wound) significantly decreased wound width, as compared to both diluent-control wounds and naïve control wounds (p<0.05), when incisional wounds were assessed at three days after wounding.

The results provided clearly illustrate that WNT3A, and hence therapeutically effective fragments or derivatives of WNT3A, may be used to accelerate wound healing.

“Sequence Information”

Sequence ID No. 1 Human WNT3A amino acid sequence
MAPLGYFLLLCSLKQALGSYPIWWSLAVGPQYSSLGSQPILCASIPGLVPKQLRFCRNYVEIMPSVAEGIKI
GIQECQHQFRGRRWNCTTVHDSLAIFGPVLDKATRESAFVHAIASAGVAFAVTRSCAEGTAAICGCSSRHQG
SPGKGWKWGGCSEDIEFGGMVSREFADARENRPDARSAMNRHNNEAGRQAIASHMHLKCKCHGLSGSCEVKT
CWWSQPDFRAIGDFLICDKYDSASEMVVEICHRESRGWVETLRPRYTYFICVPTERDLVYYEASPNFCEPNP
ETGSFGTRDRTCNVSSHGIDGCDLLCCGRGHNARAERRREKCRCVFHWCCYVSCQECTRVYDVHTCK
Sequence ID No. 2 Human WNT3A nucleotide sequence

| | |
|---|---|
| 1 | agctcccagg gcccggcccc ccccggcgct cacgctctcg gggcggactc ccggccctcc |
| 61 | gcgccctctc gcgcggcgat ggccccactc ggatacttct tactcctctg cagcctgaag |
| 121 | caggctctgg gcagctaccc gatctggtgg tcgctggctg ttgggccaca gtattcctcc |
| 181 | ctgggctcgc agcccatcct gtgtgccagc atcccgggcc tggtccccaa gcagctccgc |
| 241 | ttctgcagga actacgtgga gatcatgccc agcgtggccg agggcatcaa gattggcatc |
| 301 | caggagtgcc agcaccagtt ccgcggccgc cggtggaact gcaccaccgt ccacgacagc |
| 361 | ctggccatct tcgggcccgt gctggacaaa gctaccaggg agtcggcctt tgtccacgcc |
| 421 | attgcctcag ccggtgtggc ctttgcagtg acacgctcat gtgcagaagg cacggccgcc |
| 481 | atctgtggct gcagcagccg ccaccagggc tcaccaggca agggctggaa gtggggtggc |
| 541 | tgtagcgagg acatcgagtt tggtgggatg gtgtctcggg agttcgccga cgcccgggag |
| 601 | aaccggccag atgcccgctc agccatgaac cgccacaaca acgaggctgg gcgccaggcc |
| 661 | atcgccagcc acatgcacct caagtgcaag tgccacgggc tgtcgggcag ctgcgaggtg |
| 721 | aagacatgct ggtggtcgca acccgacttc cgcgccatcg gtgacttcct caaggacaag |
| 781 | tacgacagcg cctcggagat ggtggtggag aagcaccggg agtcccgcgg ctgggtggag |
| 841 | accctgcggc cgcgctacac ctacttcaag gtgcccacgg agcgcgacct ggtctactac |
| 901 | gaggcctcgc ccaacttctg cgagcccaac cctgagacgg gctccttcgg cacgcgcgac |
| 961 | cgcacctgca acgtcagctc gcacggcatc gacggctgcg acctgctgtg ctgcggccgc |
| 1021 | ggccacaacg cgcgagcgga gcggcgccgg gagaagtgcc gctgcgtgtt ccactggtgc |
| 1081 | tgctacgtca gctgccagga gtgcacgcgc gtctacgacg tgcacacctg caagtaggca |
| 1141 | ccggccgcgg ctccccctgg acggggcggg ccctgcctga gggtgggctt ttccctgggt |
| 1201 | ggagcaggac tcccacctaa acggggcagt actcctccct gggggcggga ctcctccctg |
| 1261 | ggggtggggc tcctacctgg gggcagaact cctacctgaa ggcagggctc ctccctggag |
| 1321 | ctagtgtctc ctctctggtg gctgggctgc tcctgaatga ggcggagctc caggatgggg |
| 1381 | aggggctctg cgttggcttc tccctgggga cggggctccc ctggacagag gcggggctac |
| 1441 | agattgggcg gggcttctct tgggtgggac agggcttctc ctgcgggggc gaggcccctc |
| 1501 | ccagtaaggg cgtggctctg ggtgggcggg gcactaggta ggcttctacc tgcaggcggg |
| 1561 | gctcctcctg aaggaggcgg ggctctagga tggggcacgg ctctggggta ggctgctccc |
| 1621 | tgagggcgga gcgcctcctt aggagtgggg ttttatggtg gatgaggctt cttcctggat |
| 1681 | ggggcagagc ttctcctgac cagggcaagg ccccttccac gggggctgtg gctctgggtg |
| 1741 | ggcgtggcct gcataggctc cttcctgtgg gtggggcttc tctgggacca ggctccaatg |
| 1801 | gggcggggct tctctccgcg ggtgggactc ttccctggga accgccctcc tgattaaggc |
| 1861 | gtggcttctg caggaatccc ggctccagag caggaaattc agcccaccag ccacctcatc |
| 1921 | cccaaccccc tgtaaggttc catccacccc tgcgtcgagc tgggaaggtt ccatgaagcg |
| 1981 | agtcgggtcc ccaacccgtg cccctgggat ccgagggccc ctctccaagc gcctggcttt |
| 2041 | ggaatgctcc aggcgcgccg acgcctgtgc caccccttcc tcagcctggg gtttgaccac |
| 2101 | ccacctgacc aggggcccta cctggggaaa gcctgaaggg cctcccagcc cccaacccca |
| 2161 | agaccaagct tagtcctggg agaggacagg gacttcgcag aggcaagcga ccgaggccct |
| 2221 | cccaaagagg cccgccctgc ccgggctccc acaccgtcag gtactcctgc cagggaactg |
| 2281 | gcctgctgcg ccccaggccc cgcccgtctc tgctctgctc agctgcgccc ccttctttgc |
| 2341 | agctgcccag cccctcctcc ctgccctcgg gtctccccac ctgcactcca tccagctaca |
| 2401 | ggagagatag aagcctctcg tcccgtccct ccctttcctc cgcctgtcca cagcccctta |
| 2461 | agggaaaggt aggaagagag gtccagcccc ccaggctgcc cagagctgct ggtctcattt |
| 2521 | gggggcgttc gggaggtttg gggggcatca accccccgac tgtgctgctc gcgaaggtcc |
| 2581 | cacagccctg agatgggccg gcccccttcc tggcccctca tggcgggact ggagaaatgg |
| 2641 | tccgctttcc tggagccaat ggcccggccc ctcctgactc atccgcctgg cccgggaatg |
| 2701 | aatggggagg ccgctgaacc cacccggccc atatccctgg ttgcctcatg gccagcgccc |
| 2761 | ctcagcctct gccactgtga accggctccc accctcaagg tgcggggaga agaagcggcc |
| 2821 | aggcggggcg ccccaagagc ccaaaagagg gcacaccgcc atcctctgcc tcaaattctg |
| 2881 | cgtttttggt tttaatgtta tatctgatgc tgctatatcc actgtccaac gg |

1-20. (canceled)

21. A method of accelerating wound healing, the method comprising providing a therapeutically effective amount of WNT3A, or a therapeutically effective fragment or derivative thereof, to a site where wound healing is to be accelerated.

22. A method according to claim 21, wherein the WNT3A, or the fragment or derivative, is administered to the site where wound healing is to be accelerated.

23. A method according to claim 21, wherein the site where wound healing is to be accelerated is a wound.

24. A method according to claim 21, wherein the site where wound healing is to be accelerated is a site where a wound is to be formed.

25. A method according to claim 21, wherein the wound, healing of which is to be accelerated, is a skin wound.

26. A method according to claim 21, wherein the wound, healing of which is to be accelerated, is an eye wound.

27. A method according to claim 21, wherein the wound, healing of which is to be accelerated, is an acute wound.

28. A method according to claim 21, wherein the wound, healing of which is to be accelerated, is a chronic wound.

29. A method according to claim 21, wherein up to 750 ng of WNT3A, or the fragment or derivative, is provided per centimetre of wound the healing of which is to be accelerated.

30. A method according to claim 21, wherein up to 18 pmoles of WNT3A, or the fragment or derivative, is provided per centimetre of wound the healing of which is to be accelerated.

31. A method according to claim 21, wherein approximately 100 ng of WNT3A, or the fragment or derivative, is provided per centimetre of wound the healing of which is to be accelerated.

32. A method according to claim 21, wherein WNT3A is provided to a site where wound healing is to be accelerated.

33. A method according to claim 21, wherein a derivative of WNT3A is provided to a site where wound healing is to be accelerated.

34. A method according to claim 21, wherein a derivative of WNT3A having increased resistance to degradation compared to WNT3A is provided to a site where wound healing is to be accelerated.

35. A method according to claim 21, wherein a peptoid derivative of WNT3A is provided to a site where wound healing is to be accelerated.

36. A method according to claim 21, wherein the WNT3A, or the fragment or derivative, is provided to the site where wound healing is to be accelerated by a topical medicament.

37. A method according to claim 21, wherein the WNT3A, or the fragment or derivative, is provided to the site where wound healing is to be accelerated by an injectable medicament.

38. A method according to claim 21, wherein the WNT3A, or the fragment or derivative, is provided to the site where wound healing is to be accelerated by an intradermal injection.


SEQUENCE LISTING (12 sequences)

SEQ ID NO: 1 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 2 - DNA, 2932 bp, Homo sapiens
agctcccagggcccggccccccccggcgctcacgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatctgatgctgctatatccactgtccaacgg

SEQ ID NO: 3 - protein, 352 aa, Mus musculus
MAPLGYLLVLCSLKQALGSYPIWWSLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK

SEQ ID NO: 4 - DNA, 2814 bp, Mus musculus
gaattcatgtcttacggtcaaggcagagggcccagcgccactgcagccgcgccacctcccagggccgggccagcccaggcgtccgcgctctcggggtggactccccccgctgcgcgctcaagccggcgatggctcctctcggatacctcttagtgctctgcagcctgaagcaggctctgggcagctacccgatctggtggtccttggctgtgggaccccagtactcctctctgagcactcagcccattctctgtgccagcatcccaggcctggtaccgaagcagctgcgcttctgcaggaactacgtggagatcatgcccagcgtggctgagggtgtcaaagcgggcatccaggagtgccagcaccagttccgaggccggcgttggaactgcaccaccgtcagcaacagcctggccatctttggccctgttctggacaaagccacccgggagtcagcctttgtccatgccatcgcctccgctggagtagctttcgcagtgacacgctcctgtgcagagggatcagctgctatctgtgggtgcagcagccgcctccagggctccccaggcgagggctggaagtggggcggctgtagtgaggacattgaatttggaggaatggtctctcgggagtttgccgatgccagggagaaccggccggatgcccgctctgccatgaaccgtcacaacaatgaggctgggcgccaggccatcgccagtcacatgcacctcaagtgcaaatgccacgggctatctggcagctgtgaagtgaagacctgctggtggtcgcagccggacttccgcaccatcggggatttcctcaaggacaagtatgacagtgcctcggagatggtggtagagaaacaccgagagtctcgtggctgggtggagaccctgaggccacgttacacgtacttcaaggtgccgacagaacgcgacctggtctactacgaggcctcacccaacttctgcgaacctaaccccgaaaccggctccttcgggacgcgtgaccgcacctgcaatgtgagctcgcatggcatagatgggtgcgacctgttgtgctgcgggcgcgggcataacgcgcgcactgagcgacggagggagaaatgccactgtgttttccattggtgctgctacgtcagctgccaggagtgcacacgtgtctatgacgtgcacacctgcaagtaggagagctcctaacacgggagcagggttcattccgaggggcaaggttcctacctgggggcggggttcctacttggaggggtctcttacttggggactcggttcttacttgagggcggagatcctacctgtgagggtctcatacctaaggacccggtttctgccttcagcctgggctcctatttgggatctgggttcctttttaggggagaagctcctgtctgggatacgggtttctgcccgagggtggggctccacttggggatggaattccaatttgggccggaagtcctacctcaatggcttggactcctctcttgacccgacagggctcaaatggagacaggtaagctactccctcaactaggtggggttcgtgcggatgggtgggaggggagagattagggtccctcctcccagaggcactgctctatctagatacatgagagggtgcttcagggtgggccctatttgggcttgaggatcccgtgggggcggggcttcaccccgactgggtggaacttttggagacccccttccactggggcaaggcttcactgaagactcatgggatggagctccacggaaggaggagttcctgagcgagcctgggctctgagcaggccatccagctcccatctggcccctttccagtcctggtgtaaggttcaacctgcaagcctcatctgcgcagagcaggatctcctggcagaatgaggcatggagaagaactcaggggtgataccaagacctaacaaaccccgtgcctgggtacctcttttaaagctctgcaccccttcttcaagggctttcctagtctccttggcagagctttcctgaggaagatttgcagtcccccagagttcaagtgaacacccatagaacagaacagactctatcctgagtagagagggttctctaggaatctctatggggactgctaggaaggatcctgggcatgacagcctcgtatgatagcctgcatccgctctgacacttaatactcagatctcccgggaaacccagctcatccggtccgtgatgtccatgccccaaatgcctcagagatgttgcctcactttgagttgtatgaacttcggagacatggggacacagtcaagccgcagagccagggttgtttcaggacccatctgattccccagagcctgctgttgaggcaatggtcaccagatccgttggccaccaccctgtcccgagcttctctagtgtctgtctggcctggaagtgaggtgctacatacagcccatctgccacaagagcttcctgattggtaccactgtgaaccgtccctccccctccagacaggggaggggatgtggccatacaggagtgtgcccggagagcgcggaaagaggaagagaggctgcacacgcgtggtgactgactgtcttctgcctggaactttgcgttcgcgcttgtaactttattttcaatgctgctatatccacccaccactggatttagacaaaagtgattttctttttttttttttcttttctttctatgaaagaaattattttagtttatagtatgtttgtttcaaataatggggaaagtaaaaagagagaaaaaaaaaaaaaaaaaaaaaaaaaaaaaaaaaaaaaaaaaa

SEQ ID NO: 5 - protein, 352 aa, Rattus sp.
MAPLGYLLELCSLKQALGSYPVWWSLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK

SEQ ID NO: 6 - DNA, 1384 bp, Rattus sp.
atggacgaaaggagcatcaacacttccaagaacaagagacaggatgtggcagtgctagcggggcactggcctcggcccgccgcccggccgccggcccacctggcgcagcgccgccctcggagcccgtgtaccggtgcacacccgggaaccccgcgcaccccgctgccacagagggcccagcgccactgcagccgcgccacctcccagggccgggccagccccggcgtacgcgctctcggggtggactccccccgctgcgcgttcaagcccacgatggctcctctcggatacctgttagagctctgcagcctgaagcaggcgctgggcagctaccctgtgtggtggtccttggctgtgggaccccagtactcctcactgagcactcagcccattctctgtgccagcatcccgggtctggtgcccaagcagctgcgcttctgcaggaactacgtggagatcatgcccagtgtggccgagggtgtcaaggcgggcatccaagagtgccagcaccagttccgaggccggcgttggaactgcaccactgtcagcaacagcctggccatctttggccccgttctggacaaagccacccgggagtcagcctttgtccatgccatcgcttccgctggagtggccttcgcagtgacccggtcctgtgcagagggatcagctgccatctgtgggtgcagcagccgcttgcagggctccccaggcgagggctggaagtggggtggctgtagtgaggacattgaatttggaggaatggtctctcgggagtttgccgatgccagggagaaccggccggatgcccgctctgccatgaaccgtcacaacaatgaggctgggcgacaggccatcgccagtcacatgcacctcaagtgcaaatgccacggactatccggcagttgcgaagtgaagacctgctggtggtcgcagcctgacttccgcaccatcggggatttcctcaaggacaagtatgacagcgcctcagagatggtggtagagaaacaccgagagtctcgtggctgggtggagaccttgaggccacgttacacatacttcaaggtgcccacagagcgcgacctggtctactacgaggcctcacctaacttctgcgagcccaaccctgaaaccggctccttcgggacgcgtgaccgcacctgcaatgtgagctcgcatggcatagacgggtgcgacctgttgtgctgcgggcgtgggcataacgcgcgcactgagcgacggagggagaaatgccactgtgttttccactggtgctgttatgtcagctgccaggagtgcacacgtgtctatgacgtgcacacctgcaagtaggagggctcctaacagagggagcagggttcattcctcggggcaagattcctat

SEQ ID NO: 7 - DNA, 5100 bp, Homo sapiens
atggagcccgagtgagcgcggcgcgggcccgtccggccgccggacaacatggaggcagcgccgcccgggccgccgtggccgctgctgctgctgctgctgctgctgctggcgctgtgcggctgcccggcccccgccgcggcctcgccgctcctgctatttgccaaccgccgggacgtacggctggtggacgccggcggagtcaagctggagtccaccatcgtggtcagcggcctggaggatgcggccgcagtggacttccagttttccaagggagccgtgtactggacagacgtgagcgaggaggccatcaagcagacctacctgaaccagacgggggccgccgtgcagaacgtggtcatctccggcctggtctctcccgacggcctcgcctgcgactgggtgggcaagaagctgtactggacggactcagagaccaaccgcatcgaggtggccaacctcaatggcacatcccggaaggtgctcttctggcaggaccttgaccagccgagggccatcgccttggaccccgctcacgggtacatgtactggacagactggggtgagacgccccggattgagcgggcagggatggatggcagcacccggaagatcattgtggactcggacatttactggcccaatggactgaccatcgacctggaggagcagaagctctactgggctgacgccaagctcagcttcatccaccgtgccaacctggacggctcgttccggcagaaggtggtggagggcagcctgacgcaccccttcgccctgacgctctccggggacactctgtactggacagactggcagacccgctccatccatgcctgcaacaagcgcactggggggaagaggaaggagatcctgagtgccctctactcacccatggacatccaggtgctgagccaggagcggcagcctttcttccacactcgctgtgaggaggacaatggcggctgctcccacctgtgcctgctgtccccaagcgagcctttctacacatgcgcctgccccacgggtgtgcagctgcaggacaacggcaggacgtgtaaggcaggagccgaggaggtgctgctgctggcccggcggacggacctacggaggatctcgctggacacgccggactttaccgacatcgtgctgcaggtggacgacatccggcacgccattgccatcgactacgacccgctagagggctatgtctactggacagatgacgaggtgcgggccatccgcagggcgtacctggacgggtctggggcgcagacgctggtcaacaccgagatcaacgaccccgatggcatcgcggtcgactgggtggcccgaaacctctactggaccgacacgggcacggaccgcatcgaggtgacgcgcctcaacggcacctcccgcaagatcctggtgtcggaggacctggacgagccccgagccatcgcactgcaccccgtgatgggcctcatgtactggacagactggggagagaaccctaaaatcgagtgtgccaacttggatgggcaggagcggcgtgtgctggtcaatgcctccctcgggtggcccaacggcctggccctggacctgcaggaggggaagctctactggggagacgccaagacagacaagatcgaggtgatcaatgttgatgggacgaagaggcggaccctcctggaggacaagctcccgcacattttcgggttcacgctgctgggggacttcatctactggactgactggcagcgccgcagcatcgagcgggtgcacaaggtcaaggccagccgggacgtcatcattgaccagctgcccgacctgatggggctcaaagctgtgaatgtggccaaggtcgtcggaaccaacccgtgtgcggacaggaacggggggtgcagccacctgtgcttcttcacaccccacgcaacccggtgtggctgccccatcggcctggagctgctgagtgacatgaagacctgcatcgtgcctgaggccttcttggtcttcaccagcagagccgccatccacaggatctccctcgagaccaataacaacgacgtggccatcccgctcacgggcgtcaaggaggcctcagccctggactttgatgtgtccaacaaccacatctactggacagacgtcagcctgaagaccatcagccgcgccttcatgaacgggagctcggtggagcacgtggtggagtttggccttgactaccccgagggcatggccgttgactggatgggcaagaacctctactgggccgacactgggaccaacagaatcgaagtggcgcggctggacgggcagttccggcaagtcctcgtgtggagggacttggacaacccgaggtcgctggccctggatcccaccaagggctacatctactggaccgagtggggcggcaagccgaggatcgtgcgggccttcatggacgggaccaactgcatgacgctggtggacaaggtgggccgggccaacgacctcaccattgactacgctgaccagcgcctctactggaccgacctggacaccaacatgatcgagtcgtccaacatgctgggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcctggtgttccactcctcccgccaggatggcctcaatgactgtatgcacaacaacgggcagtgtgggcagctgtgccttgccatccccggcggccaccgctgcggctgcgcctcacactacaccctggaccccagcagccgcaactgcagcccgcccaccaccttcttgctgttcagccagaaatctgccatcagtcggatgatcccggacgaccagcacagcccggatctcatcctgcccctgcatggactgaggaacgtcaaagccatcgactatgacccactggacaagttcatctactgggtggatgggcgccagaacatcaagcgagccaaggacgacgggacccagccctttgttttgacctctctgagccaaggccaaaacccagacaggcagccccacgacctcagcatcgacatctacagccggacactgttctggacgtgcgaggccaccaataccatcaacgtccacaggctgagcggggaagccatgggggtggtgctgcgtggggaccgcgacaagcccagggccatcgtcgtcaacgcggagcgagggtacctgtacttcaccaacatgcaggaccgggcagccaagatcgaacgcgcagccctggacggcaccgagcgcgaggtcctcttcaccaccggcctcatccgccctgtggccctggtggtagacaacacactgggcaagctgttctgggtggacgcggacctgaagcgcattgagagctgtgacctgtcaggggccaaccgcctgaccctggaggacgccaacatcgtgcagcctctgggcctgaccatccttggcaagcatctctactggatcgaccgccagcagcagatgatcgagcgtgtggagaagaccaccggggacaagcggactcgcatccagggccgtgtcgcccacctcactggcatccatgcagtggaggaagtcagcctggaggagttctcagcccacccatgtgcccgtgacaatggtggctgctcccacatctgtattgccaagggtgatgggacaccacggtgctcatgcccagtccacctcgtgctcctgcagaacctgctgacctgtggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggcggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgtgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctgacctcggccgggccactctggcttctctgtgcccctgtaaatagttttaaatatgaacaaagaaaaaaatatattttatgatttaaaaaataaatataattgggattttaaaaacatgagaaatgtgaactgtgatggggtgggcagggctgggagaactttgtacagtggaacaaatatttataaacttaattttgtaaaacag

SEQ ID NO: 8 - protein, 1610 aa, Homo sapiens
MEAAPPGPPWPLLLLLLLLLALCGCPAPAAASPLLLFANRRDVRLVDAGGVKLESTIVVSGLEDAAAVDFQFSKGAVYWTDVSEEAIKQTYLNQTGAAVQNVVISGLVSPDGLACDWVGKKLYWTDSETNRIEVANLNGTSRKVLFWQDLDQPRAIALDPAHGYMYWTDWGETPRIERAGMDGSTRKIIVDSDIYWPNGLTIDLEEQKLYWADAKLSFIHRANLDGSFRQKVVEGSLTHPFALTLSGDTLYWTDWQTRSIHACNKRTGGKRKEILSALYSPMDIQVLSQERQPFFHTRCEEDNGGCSHLCLLSPSEPFYTCACPTGVQLQDNGRTCKAGAEEVLLLARRTDLRRISLDTPDFTDIVLQVDDIRHAIAIDYDPLEGYVYWTDDEVRAIRRAYLDGSGAQTLVNTEINDPDGIAVDWVARNLYWTDTGTDRIEVTRLNGTSRKILVSEDLDEPRAIALHPVMGLMYWTDWGENPKIECANLDGQERRVLVNASLGWPNGLALDLQEGKLYWGDAKTDKIEVINVDGTKRRTLLEDKLPHIFGFTLLGDFIYWTDWQRRSIERVHKVKASRDVIIDQLPDLMGLKAVNVAKVVGTNPCADRNGGCSHLCFFTPHATRCGCPIGLELLSDMKTCIVPEAFLVFTSRAAIHRISLETNNNDVAIPLTGVKEASALDFDVSNNHIYWTDVSLKTISRAFMNGSSVEHVVEFGLDYPEGMAVDWMGKNLYWADTGTNRIEVARLDGQFRQVLVWRDLDNPRSLALDPTKGYIYWTEWGGKPRIVRAFMDGTNCMTLVDKVGRANDLTIDYADQRLYWTDLDTNMIESSNMLGQERVVIADDLPHPFGLTQYSDYIYWTDWNLHSIERADKTSGRNRTLIQGHLDFVMDILVFHSSRQDGLNDCMHNNGQCGQLCLAIPGGHRCGCASHYTLDPSSRNCSPPTTFLLFSQKSAISRMIPDDQHSPDLILPLHGLRNVKAIDYDPLDKFIYWVDGRQNIKRAKDDGTQPFVLTSLSQGQNPDRQPHDLSIDIYSRTLFWTCEATNTINVHRLSGEAMGVVLRGDRDKPRAIVVNAERGYLYFTNMQDRAAKIERAALDGTEREVLFTTGLIRPVALVVDNTLGKLFWVDADLKRIESCDLSGANRLTLEDANIVQPLGLTILGKHLYWIDRQQQMIERVEKTTGDKRTRIQGRVAHLTGIHAVEEVSLEEFSAHPCARDNGGCSHICIAKGDGTPRCSCPVHLVLLQNLLTCGEPPTCSPDQFACATGEIDCIPGAWRCDGFPECDDQSDEEGCPVCSAAQFPCARGQCVDLRLRCDGEADCQDRSDEADCDAICLPNQFRCASGQCVLIKQQCDSFPDCIDGSDELMCEITKPPSDDSPAHSSAIGPVIGIILSLFVMGGVYFVCQRVVCQRYAGANGPFPHEYVSGTPHVPLNFIAPGGSQHGPFTGIACGKSMMSSVSLMGGRGGVPLYDRNHVTGASSSSSSSTKATLYPPILNPPPSPATDPSLYNMDMFYSSNIPATVRPYRPYIIRGMAPPTTPCSTDVCDSDYSASRWKASKYYLDLNSDSDPYPPPPTPHSQYLSAEDSCPPSPATERSYFHLFPPPPSP

SEQ ID NO: 9 - DNA, 5301 bp, Homo sapiens
gcggccgccccggctcctcgcctcccccacttctggccacccctcgccggtgagagaagagaacgcgagaagggaagatgggggccgtcctgaggagcctcctggcctgcagcttctgtgtgctcctgagagcggcccctttgttgctttatgcaaacagacgggacttgcgattggttgatgctacaaatggcaaagagaatgctacgattgtagttggaggcttggaggatgcagctgcggtggactttgtgtttagtcatggcttgatatactggagtgatgtcagcgaagaagccattaaacgaacagaatttaacaaaactgagagtgtgcagaatgttgttgtttctggattattgtcccccgatgggctggcatgtgattggcttggagaaaaattgtactggacagattctgaaactaatcggattgaagtttctaatttagatggatctttacgaaaagttttattttggcaagagttggatcaacccagagctattgccttagatccttcaagtgggttcatgtactggacagactggggagaagtgccaaagatagaacgtgctggaatggatggttcaagtcgcttcattataataaacagtgaaatttactggccaaatggactgactttggattatgaagaacaaaagctttattgggcagatgcaaaacttaatttcatccacaaatcaaatctggatggaacaaatcggcaggcagtggttaaaggttcccttccacatccttttgccttgacgttatttgaggacatattgtactggactgactggagcacacactccattttggcttgcaacaagtatactggtgagggtctgcgtgaaatccattctgacatcttctctcccatggatatacatgccttcagccaacagaggcagccaaatgccacaaatccatgtggaattgacaatgggggttgttcccatttgtgtttgatgtctccagtcaagcctttttatcagtgtgcttgccccactggggtcaaactcctggagaatggaaaaacctgcaaagatggtgccacagaattattgcttttagctcgaaggacagacttgagacgcatttctttggatacaccagattttacagacattgttctgcagttagaagacatccgtcatgccattgccatagattacgatcctgtggaaggctacatctactggactgatgatgaagtgagggccatacgccgttcatttatagatggatctggcagtcagtttgtggtcactgctcaaattgcccatcctgatggtattgctgtggactgggttgcacgaaatctttattggacagacactggcactgatcgaatagaagtgacaaggctcaatgggaccatgaggaagatcttgatttcagaggacttagaggaaccccgggctattgtgttagatcccatggttgggtacatgtattggactgactggggagaaattccgaaaattgagcgagcagctctggatggttctgaccgtgtagtattggttaacacttctcttggttggccaaatggtttagccttggattatgatgaaggcaaaatatactggggagatgccaaaacagacaagattgaggttatgaatactgatggcactgggagacgagtactagtggaagacaaaattcctcacatatttggatttactttgttgggtgactatgtttactggactgactggcagaggcgtagcattgaaagagttcataaacgaagtgcagagagggaagtgatcatagatcagctgcctgacctcatgggcctaaaggctacaaatgttcatcgagtgattggttccaacccctgtgctgaggaaaacgggggatgtagccatctctgcctctatagacctcagggccttcgctgtgcttgccctattggctttgaactcatcagtgacatgaagacctgcattgtcccagaggctttccttttgttttcacggagagcagatatcagacgaatttctctggaaacaaacaataataatgtggctattccactcactggtgtcaaagaagcttctgctttggattttgatgtgacagacaaccgaatttattggactgatatatcactcaagaccatcagcagagcctttatgaatggcagtgcactggaacatgtggtagaattcggcttagattatccagaaggcatggcagtagactggcttgggaagaacttgtactgggcagacacaggaacgaatcgaattgaggtgtcaaagttggatgggcagcaccgacaagttttggtgtggaaagacctagatagtcccagagctctcgcgttggaccctgccgaaggatttatgtattggactgaatggggtggaaaacctaagatagacagagctgcaatggatggaagtgaacgtactaccttagttccaaatgtggggcgggcaaacggcctaactattgattatgctaaaaggaggctttattggacagacctggacaccaacttaatagaatcttcaaatatgcttgggctcaaccgtgaagttatagcagatgacttgcctcatccttttggcttaactcagtaccaagattatatctactggacggactggagccgacgcagcattgagcgtgccaacaaaaccagtggccaaaaccgcaccatcattcagggccatttggattatgtgatggacatcctcgtctttcactcatctcgacagtcagggtggaatgaatgtgcttccagcaatgggcactgctcccacctctgcttggctgtgccagttgggggttttgtttgtggatgccctgcccactactctcttaatgctgacaacaggacttgtagtgctcctacgactttcctgctcttcagtcaaaagagtgccatcaaccgcatggtgattgatgaacaacagagccccgacatcatccttcccatccacagccttcggaatgtccgggccattgactatgacccactggacaagcaactctattggattgactcacgacaaaacatgatccgaaaggcacaagaagatggcagccagggctttactgtggttgtgagctcagttccgagtcagaacctggaaatacaaccctatgacctcagcattgatatttacagccgctacatctactggacttgtgaggctaccaatgtcattaatgtgacaagattagatgggagatcagttggagtggtgctgaaaggcgagcaggacagacctcgagccattgtggtaaacccagagaaagggtatatgtattttaccaatcttcaggaaaggtctcctaaaattgaacgggctgctttggatgggacagaacgggaggtcctctttttcagtggcttaagtaaaccaattgctttagcccttgatagcaggctgggcaagctcttttgggctgattcagatctccggcgaattgaaagcagtgatctctcaggtgctaaccggatagtattagaagactccaatatcttgcagcctgtgggacttactgtgtttgaaaactggctctattggattgataaacagcagcaaatgattgaaaaaattgacatgacaggtcgagagggtagaaccaaagtccaagctcgaattgcccagcttagtgacattcatgcagtaaaggagctgaaccttcaagaatacagacagcacccttgtgctcaggataatggtggctgttcacatatttgtcttgtaaagggggatggtactacaaggtgttcttgccccatgcacctggttctacttcaagatgagctatcatgtggagaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctgaggaggggccctcctcctctgactgcctccaacgtaaaaatgtaaatataaatttggttgagatctggagggggggagggagctattagagaaggatgaggcagaccatgtacagttaaaattataaaatggggtagggaatactggagatatttgtacagaagaaaaggatatttatatattttcttaaaacagcagatttgctgcttgtgccataaaagtttgtataaaaaaaatttgtactaaaagttttatttttgcaaactaaatacacaaagcatgccttaaacccagtgaagcaactgagtacaaaggaaacaggaataataaaggcatcactgaccaggaatatctgggctttattgataccaaaaaaaaaaaaa

SEQ ID NO: 10 - protein, 1613 aa, Homo sapiens
MGAVLRSLLACSFCVLLRAAPLLLYANRRDLRLVDATNGKENATIVVGGLEDAAAVDFVFSHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSDIFSPMDIHAFSQQRQPNATNPCGIDNGGCSHLCLMSPVKPFYQCACPTGVKLLENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGKIYWGDAKTDKIEVMNTDGTGRRVLVEDKIPHIFGFTLLGDYVYWTDWQRRSIERVHKRSAEREVIIDQLPDLMGLKATNVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQSGWNECASSNGHCSHLCLAVPVGGFVCGCPAHYSLNADNRTCSAPTTFLLFSQKSAINRMVIDEQQSPDIILPIHSLRNVRAIDYDPLDKQLYWIDSRQNMIRKAQEDGSQGFTVVVSSVPSQNLEIQPYDLSIDIYSRYIYWTCEATNVINVTRLDGRSVGVVLKGEQDRPRAIVVNPEKGYMYFTNLQERSPKIERAALDGTEREVLFFSGLSKPIALALDSRLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 11 - DNA, 3195 bp, Homo sapiens
acagcatggagtggggttacctgttggaagtgacctcgctgctggccgccttggcgctgctgcagcgctctagcggcgctgcggccgcctcggccaaggagctggcatgccaagagatcaccgtgccgctgtgtaagggcatcggctacaactacacctacatgcccaatcagttcaaccacgacacgcaagacgaggcgggcctggaggtgcaccagttctggccgctggtggagatccagtgctcgcccgatctcaagttcttcctgtgcagcatgtacacgcccatctgcctagaggactacaagaagccgctgccgccctgccgctcggtgtgcgagcgcgccaaggccggctgcgcgccgctcatgcgccagtacggcttcgcctggcccgaccgcatgcgctgcgaccggctgcccgagcaaggcaaccctgacacgctgtgcatggactacaaccgcaccgacctaaccaccgccgcgcccagcccgccgcgccgcctgccgccgccgccgcccggcgagcagccgccttcgggcagcggccacggccgcccgccgggggccaggcccccgcaccgcggaggcggcaggggcggtggcggcggggacgcggcggcgcccccagctcgcggcggcggcggtggcgggaaggcgcggccccctggcggcggcgcggctccctgcgagcccgggtgccagtgccgcgcgcctatggtgagcgtgtccagcgagcgccacccgctctacaaccgcgtcaagacaggccagatcgctaactgcgcgctgccctgccacaacccctttttcagccaggacgagcgcgccttcaccgtcttctggatcggcctgtggtcggtgctctgcttcgtgtccaccttcgccaccgtctccaccttccttatcgacatggagcgcttcaagtacccggagcggcccattatcttcctctcggcctgctacctcttcgtgtcggtgggctacctagtgcgcctggtggcgggccacgagaaggtggcgtgcagcggtggcgcgccgggcgcggggggcgctgggggcgcgggcggcgcggcggcgggcgcgggcgcggcgggcgcgggcgcgggcggcccgggcgggcgcggcgagtacgaggagctgggcgcggtggagcagcacgtgcgctacgagaccaccggccccgcgctgtgcaccgtggtcttcttgctggtctacttcttcggcatggccagctccatctggtgggtgatcttgtcgctcacatggttcctggcggccggtatgaagtggggcaacgaagccatcgccggctactcgcagtacttccacctggccgcgtggcttgtgcccagcgtcaagtccatcgcggtgctggcgctcagctcggtggacggcgacccggtggcgggcatctgctacgtgggcaaccagagcctggacaacctgcgcggcttcgtgctggcgccgctggtcatctacctcttcatcggcaccatgttcctgctggccggcttcgtgtccctgttccgcatccgctcggtcatcaagcaacaggacggccccaccaagacgcacaagctggagaagctgatgatccgcctgggcctgttcaccgtgctctacaccgtgcccgccgcggtggtggtcgcctgcctcttctacgagcagcacaaccgcccgcgctgggaggccacgcacaactgcccgtgcctgcgggacctgcagcccgaccaggcacgcaggcccgactacgccgtcttcatgctcaagtacttcatgtgcctagtggtgggcatcacctcgggcgtgtgggtctggtccggcaagacgctggagtcctggcgctccctgtgcacccgctgctgctgggccagcaagggcgccgcggtgggcgggggcgcgggcgccacggccgcggggggtggcggcgggccggggggcggcggcggcgggggacccggcggcggcggggggccgggcggcggcgggggctccctctacagcgacgtcagcactggcctgacgtggcggtcgggcacggcgagctccgtgtcttatccaaagcagatgccattgtcccaggtctgagcggaggggagggggcgcccaggaggggtggggaggggggcgaggagacccaagtgcagcgaagggacacttgatgggctgaggttcccaccccttcacagtgttgattgctattagcatgataatgaactcttaatggtatccattagctgggacttaaatgactcacttagaacaaagtacctggcattgaagcctcccagacccagccccttttcctccattgatgtgcggggagctcctcccgccacgcgttaatttctgttggctgaggagggtggactctgcggcgtttccagaacccgagatttggagccctccctggctgcacttggctgggtttgcagtcagatacacagatttcacctgggagaacctctttttctccctcgactcttcctacgtaaactcccacccctgacttaccctggaggaggggtgaccgccacctgatgggattgcacggtttgggtattcttaatgaccaggcaaatgccttaagtaaacaaacaagaaatgtcttaattatacaccccacgtaaatacgggtttcttacattagaggatgtatttatataattatttgttaaattgtaaaaaaaaaaagtgtaaaatatgtatatatccaaagatatagtgtgtacatttttttgtaaaaagtttagaggcttacccctgtaagaacagatataagtattctattttgtcaataaaatgacttttgataaatgatttaaccattgccctctcccccgcctcttctgagctgtcacctttaaagtgcttgctaaggacgcatggggaaaatggacattttctggcttgtcattctgtacactgaccttaggcatggagaaaattacttgttaaactctagttcttaagttgttagccaagtaaatatcattgttgaactgaaatcaaaattgagtttttgcaccttccccaaagacggtgtttttcatgggagctcttttctgatccatggataacaactctcactttagtggatgtaaatggaacttctgcaaggcagtaattccccttaggccttgttatttatcctgcatggtatcactaaaggtttcaaaaccctgaaaaaaaa

SEQ ID NO: 12 - protein, 694 aa, Homo sapiens
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTAAPSPPRRLPPPPPGEQPPSGSGHGRPPGARPPHRGGGRGGGGGDAAAPPARGGGGGGKARPPGGGAAPCEPGCQCRAPMVSVSSERHPLYNRVKTGQIANCALPCHNPFFSQDERAFTVFWIGLWSVLCFVSTFATVSTFLIDMERFKYPERPIIFLSACYLFVSVGYLVRLVAGHEKVACSGGAPGAGGAGGAGGAAAGAGAAGAGAGGPGGRGEYEELGAVEQHVRYETTGPALCTVVFLLVYFFGMASSIWWVILSLTWFLAAGMKWGNEAIAGYSQYFHLAAWLVPSVKSIAVLALSSVDGDPVAGICYVGNQSLDNLRGFVLAPLVIYLFIGTMFLLAGFVSLFRIRSVIKQQDGPTKTHKLEKLMIRLGLFTVLYTVPAAVVVACLFYEQHNRPRWEATHNCPCLRDLQPDQARRPDYAVFMLKYFMCLVVGITSGVWVWSGKTLESWRSLCTRCCWASKGAAVGGGAGATAAGGGGGPGGGGGGGPGGGGGPGGGGGSLYSDVSTGLTWRSGTASSVSYPKQMPLSQV